SEQUENCE LISTING (31 sequences)

SEQ ID NO: 1 - DNA, 25 bp, Photinus pyralis
ttatgtttttggcgtcttccatggt

SEQ ID NO: 2 - DNA, 26 bp, Photinus pyralis
gccaaaaacataaaccatggaagact

SEQ ID NO: 3 - DNA, 25 bp, Photinus pyralis
cctcttacctcagttacaatttata

SEQ ID NO: 4 - DNA, 21 bp, Photinus pyralis
cuuacgcugaguacuucgatt

SEQ ID NO: 5 - DNA, 21 bp, Photinus pyralis
ucgaaguacucagcguaagtt

SEQ ID NO: 6 - DNA, 21 bp, Escherichia coli
gcgauaagucgugucuuactt

SEQ ID NO: 7 - DNA, 21 bp, Escherichia coli
guaagacacgacuuaucgctt

SEQ ID NO: 8 - DNA, 21 bp, Homo sapiens
agggcaacuuccagaccautt

SEQ ID NO: 9 - DNA, 21 bp, Homo sapiens
auggucuggaaguugcccutt

SEQ ID NO: 10 - DNA, 21 bp, Mus musculus
cacucagucucuaagggcutt

SEQ ID NO: 11 - DNA, 21 bp, Mus musculus
agcccuuagagacugagugtt

SEQ ID NO: 12 - DNA, 21 bp, Aequorea victoria
gaacggcaucaaggugaactt

SEQ ID NO: 13 - DNA, 21 bp, Aequorea victoria
guucaccuugaucccguuctt

SEQ ID NO: 14 - DNA, 21 bp, Variola virus
cgggauaucuccagacggatt

SEQ ID NO: 15 - DNA, 21 bp, Variola virus
uccgucuggagauaucccgtt

SEQ ID NO: 16 - DNA, 20 bp, Mus musculus
aggcttcgtggattctcttg

SEQ ID NO: 17 - DNA, 21 bp, Mus musculus
cctctatatccgtttccagtc

SEQ ID NO: 18 - DNA, 22 bp, Mus musculus
atcatcttgtgaaacagcagtg

SEQ ID NO: 19 - DNA, 21 bp, Mus musculus
gcatcagcttggacaaggtgt

SEQ ID NO: 20 - DNA, 71 bp, Mus musculus
ggagctttgggaagaggaaggtgtcatccttcctgtcagatggcatcttcctcttcccgaagctccttttt

SEQ ID NO: 21 - DNA, 72 bp, Mus musculus
ggattccaattcagcgggagccacctgatgaagcttgatcgggtggctctcgctgagttggaatccattttt

SEQ ID NO: 22 - DNA, 20 bp, Mus musculus
gggaacagccaccattgttg

SEQ ID NO: 23 - DNA, 20 bp, Mus musculus
gggaagtaaggaccagagac

SEQ ID NO: 24 - DNA, 21 bp, Photinus pyralis
aacuuacgcugaguacuucga

SEQ ID NO: 25 - DNA, 21 bp, Mus musculus
acauugucacugcuaucuatt

SEQ ID NO: 26 - DNA, 21 bp, Mus musculus
uagauagcagugacaaugutt

SEQ ID NO: 27 - DNA, 21 bp, Mus musculus
ccacaaaugaagacuuauatt

SEQ ID NO: 28 - DNA, 19 bp, Mus musculus
gatcggagctgcaagattc

SEQ ID NO: 29 - DNA, 21 bp, Mus musculus
uauaagucuucauuuguggtt

SEQ ID NO: 30 - DNA, 19 bp, Mus musculus
tcgggatgtcacacaatgc

SEQ ID NO: 31 - DNA, 19 bp, Mus musculus
ttgtcggtgcaatagttcc

(19) United States
(12) Patent Application Publication
(10) Pub. No.: US 2005/0037989 A1
(43) Pub. Date: Feb. 17, 2005

(54) Inhibition of gene function by delivery of polynucleotide-based gene expression inhibitors to mammalian cells in vivo

(76) Inventors: David L. Lewis, Madison, WI (US); Jon A. Wolff, Madison, WI (US); Hans Herweijer, Madison, WI (US); James E. Hagstrom, Middleton, WI (US); Aaron G. Loomis, Prairie du Sac, WI (US); Andrew G. Spencer, Madison, WI (US)

Correspondence Address: Mark K. Johnson;Mirus Corporation, 505 S. Rosa Rd., Madison, WI, 53719, US

(21) Appl. No.: 10/874,528

(22) Filed: Jun. 23, 2004

Related U.S. Application Data
(63) Continuation-in-part of application No. 10/012,804, filed on Nov. 6, 2001.
(60) Provisional application No. 60/315,394, filed on Aug. 27, 2001.
(60) Provisional application No. 60/324,155, filed on Sep. 20, 2001.

(51) Int. Cl. A61K 48/00

(52) U.S. Cl. 514/44

(57) ABSTRACT

A process is provided to deliver polynucleotide-based gene expression inhibitors to cells in a mammal in vivo for the purpose of inhibiting gene expression in the cells. Inhibition is sequence-specific and relies on sequence similaroty of the polynucleotide-based gene expression inhibitor and the target nucleic acid molecule. Delivery of the polynucleotide-based gene expression inhibitor can enhance the efficacy of co-delivered small molecule drugs.

### CROSS-REFERENCE TO RELATED INVENTIONS

This application is a continuation-in-part of application Ser. No. 10/012,804, filed Nov. 6, 2001 which is incorporated herein by reference, and claims the benefit of U.S. Provisional Application No. 60/482,195, filed Jun. 24, 2003, U.S. Provisional Application No. 60/503,834 filed Sep. 17, 2003, U.S. Provisional Application No. 60/514,850 filed Oct. 27, 2003, U.S. Provisional Application No. 60/515,532 filed Oct. 29, 2003, and U.S. Provisional Application No. 60/547,718, filed Feb. 25, 2004. Application Ser. No. 10/012,804 claims the benefit of U.S. Provisional Application No. 60/315,394 filed Aug. 27, 2001, and 60/324,155 filed Sep. 20, 2001.

### BACKGROUND OF THE INVENTION

The delivery of genetic material as a therapeutic, gene therapy, promises to be a revolutionary advance in the treatment of disease. Although, the initial motivation for gene therapy was the treatment of genetic disorders, it is becoming increasingly apparent that gene therapy will be useful for the treatment of a broad range of acquired diseases such as cancer, infectious disorders (AIDS), heart disease, arthritis, and neurodegenerative disorders (Parkinson's and Alzheimer's). Not only can functional genes be delivered to repair a genetic deficiency, but nucleic acid can also be delivered to inhibit gene expression to provide a therapeutic effect. Inhibition of gene expression can be affected by antisense polynucleotides, siRNA mediated RNA interference and ribozymes. Transfer methods currently being explored for delivering nucleic acids to cell in vivo include viral vectors and physical-chemical, or non-viral, methods.

RNA interference (RNAi) describes the phenomenon whereby the presence of double-stranded RNA (dsRNA) of sequence that is identical or highly similar to a target gene results in the degradation of messenger RNA (mRNA) transcribed from that target gene (Sharp 2001). RNAi is a natural cellular process that has recently been harnessed for a rapidly growing number of scientific, biotechnological, and therapeutic applications. In eukaryotic cells some long, double stranded RNA (dsRNA) molecules are processed into short fragments of 21-25 base pairs with two or three overhanging 3′ nucleotides on both ends. These fragments are able to initiate the sequence-specific cleavage, and thus inactivation, of single stranded RNA (ssRNA) molecules containing a homologous sequence motif (typically messenger RNA, mRNA). More recently, it has been shown that siRNAs <30 bp delivered to a cell, induce RNAi in mammalian cells in culture and in vivo (Tuschl et al. 1999; Elbashir et al. 2001). The multi-domain enzyme complexes that are thought to catalyze the silencing process reside in the cytoplasm. Thus, the siRNA also has to be in the cytoplasm in order to guide the RNA-silencing enzyme complex to the target RNA. Gene silencing can also be initiated in mammalian cells by transfection with an expression vector producing the siRNA using the cells' own transcription machinery. In this case, the transcript is generated in the nucleus and has to be efficiently exported into the cytoplasm to cause RNA interference.

There are two major approaches to initiate siRNA-mediated silencing in mammalian cells. First, synthetic siRNA duplexes (typically between 19-30 base pairs in length) can be designed and generated against any gene the sequence of which is known. There are some guidelines and software that make designing siRNAs easier. In spite of the guidelines, not all sequences are equally efficient in initiating degradation of a target mRNA. The best, most effective siRNAs have to be determined empirically. The synthetic siRNA then has to be delivered into the cytoplasm by one of various delivery methods. Second, expression cassettes that will generate siRNA within the cell can be delivered to the cell. The currently used siRNA expression cassettes take advantage of RNA Polymerase III (Pol-III) promoters, e.g., U6. Other siRNA expression vectors with RNA Polymerase II (Pol-II) promoters have also been described. Transcripts produced by RNA Polymerase-III lack the polyA tail, and have well defined transcription start and termination signals. The expression cassette can be designed to yield a short RNA resembling the synthetic siRNA with overhanging 3′ nucleotides. The two basic types of siRNA expression constructs code either for a hairpin RNA containing both the sense and the antisense sequence, separated by a loop region, or they contain two separate promoters driving the transcription of the sense and antisense RNA strand separately.

The ability to specifically inhibit expression of a target gene by RNAi has obvious benefits. For example, RNAi could be used to generate animals that mimic true genetic “knockout” animals to study gene function. In addition, many diseases arise from the abnormal expression of a particular gene or group of genes. RNAi could be used to inhibit the expression of the genes and therefore alleviate symptoms of or cure the disease. For example, genes contributing to a cancerous state could be inhibited. In addition, viral genes could be inhibited, as well as mutant genes causing dominant genetic diseases such as myotonic dystrophy. Inhibiting such genes as cyclooxygenase or cytokines could also treat inflammatory diseases such as arthritis. Nervous system disorders could also be treated. Examples of targeted organs would include the liver, pancreas, spleen, skin, brain, prostrate, heart etc. The ability to safely delivery siRNA to mammalian cells in vivo has profound potential for the treatment of infections and diseases as well as drug discovery and target validation.

Several aspects of current pharmaceutical research and therapeutic treatment are candidates for siRNA technology. For the purposes of target validation, gene inactivation allows the investigator to assess the potential therapeutic effect of inhibiting a specific gene product. Expression arrays can be used to determine the responsive effect of inhibition on the expression of genes other than the targeted gene or pathway. Other methods of gene inactivation, generation of mutant cell lines or knockout mice suffer from serious deficiencies including embryonic lethality, expense, and inflexibility. Also, these methods frequently do not adequately model larger animals. Development of a more robust and easily applicable gene inactivation technology that can be utilized in both in vitro and in vivo models would greatly expedite the drug discovery process.

A variety of methods and routes of administration have been developed to deliver pharmaceuticals that include small molecular drugs and biologically active compounds such as peptides, hormones, proteins, and enzymes to their site of action. Parenteral routes of administration include intravascular (intravenous, intra-arterial), intramuscular, intraparenchymal, intradermal, subdermal, subcutaneous, intratumor, intraperitoneal, and intralymphatic injections that use a syringe and a needle or catheter. The blood circulatory system provides systemic spread of the pharmaceutical. Polyethylene glycol and other hydrophilic polymers have provided protection of the pharmaceutical in the blood stream by preventing its interaction with blood components and to increase the circulatory time of the pharmaceutical by preventing opsonization, phagocytosis and uptake by the reticuloendothelial system. For example, the enzyme adenosine deaminase has been covalently modified with polyethylene glycol to increase the circulatory time and persistence of this enzyme in the treatment of patients with adenosine deaminase deficiency.

Transdermal routes of administration include oral, nasal, respiratory, and vaginal administration. These routes have attracted particular interest for the delivery of peptides, proteins, hormones, and cytokines, which are typically administered by parenteral routes using needles.

Non-viral vectors, such as liposomes and cationic polymers, are currently being developed to serve as gene transfer agents. Nucleic acid-containing complexes made with these vectors can be linked with proteins or other ligands for the purpose of targeting the nucleic acid to specific tissues by receptor-mediated endocytosis. It has been shown that cationic proteins like histones and protamines or synthetic polymers like polylysine, polyarginine, polyornithine, DEAE dextran, polybrene, and polyethylenimine may be effective intracellular delivery agents while small polycations like spermine are typically ineffective.

The intravascular delivery of nucleic acid has been shown to be highly effective for gene transfer into tissue in vivo (U.S. application Ser. No. 09/330,909, U.S. Pat. No. 6,627,616). Non-viral vectors are inherently safer than viral vectors, have a reduced immune response induction and have significantly lower cost of production. Furthermore, a much lower risk of transforming activity is associated with non-viral polynucleotides than with viruses.

### SUMMARY OF THE INVENTION

In a preferred embodiment we describe processes for delivering a RNA function inhibitor (hereafter referred to as “inhibitor”) to an animal cell. We also describe compositions that facilitate delivery of an inhibitor to an animal cell. Delivery of the inhibitor results in inhibition of target gene expression by causing degradation of inhibition of function of RNA. Inhibitors are selected from the group comprising siRNA, dsRNA, antisense nucleic acid, ribozymes, RNA polymerase III transcribed DNAs, microRNA, and the like. A preferred inhibitor is siRNA.

In a preferred embodiment, we describe an in vivo process for delivery of an inhibitor to a cell of a mammal for the purposes of inhibition of gene expression (RNA function) comprising: making an inhibitor, injecting the inhibitor into a vessel, and delivering the inhibitor to a cell within a tissue thereby inhibiting expression of a target gene in the cell. Permeability of the vessel to the inhibitor may comprise increasing the pressure within the vessel by rapidly injecting a large volume of fluid into the vessel and blocking the flow of fluid into and/or out of the target tissue. This increased pressure is controlled by altering the injection volume, altering the rate of volume insertion, and by constricting the flow of blood into or out of the tissue during the procedure. The volume consists of an inhibitor in a solution wherein the solution may contain a compound or compounds which may or may not complex with the inhibitor and aid in delivery.

In a preferred embodiment, a process is described for increasing the transit of the inhibitor out of a vessel and into the cells of the surrounding tissue, comprising rapidly injecting a large volume into a vessel supplying the target tissue, thus forcing fluid out of the vasculature into the extravascular space. This process is accomplished by forcing a volume containing the inhibitor into a vessel and either constricting the flow of fluid into and/or out of an area, adding a molecule that increases the permeability of a vessel, or both. The target tissue comprises the cells supplied by the vessel distal to the point of injection. For injection into arteries, the target tissue is the cells that the arteries supply with blood. For injection into veins, the target tissue is the cells from which the vein drains blood.

In a preferred embodiment, we describe a process for inhibiting gene expression in an animal cell comprising: delivering of one or more siRNAs to the cell. The siRNA comprise a sequence that is identical, nearly identical, or complementary to the same, different, or overlapping segments of a target gene sequence(s). The siRNA may be formed outside the cell and then delivered to the cell. Alternatively, the siRNA may be transcribed within the cell from of a nucleic acid that is delivered to the cell.

The siRNA may be delivered to cells in vivo, ex vivo, in situ, or in vitro. The cell can be an animal cell that is maintained in tissue culture such as cell lines that are immortalized or transformed. The cell can be a primary or secondary cell which means that the cell has been maintained in culture for a relatively short time after being obtained from an animal. The cell can also be a mammalian cell that is within a tissue in situ or in vivo meaning that the cell has not been removed from the tissue or the animal.

In a preferred embodiment the siRNA may be modified by association or attachment of a functional group. The functional group can be, but is not limited to, a transfection reagent, targeting signal or a label or other group that facilitates delivery of the inhibitor.

In a preferred embodiment, a combination of two or more inhibitors are delivered together or sequentially to enhance inhibition of target gene expression. The inhibitors comprise sequence that is identical, nearly identical, or complementary to the same, different, or overlapping segments of the target gene sequence(s). For instance, a preferred combination comprises one inhibitor that is a siRNA and another inhibitor that is an antisense polynucleotide. A preferred antisense polynucleotide is a morpholino or a 2′-O-methyl oligonucleotide. The inhibitors may be delivered to cells in vivo, ex vivo, in situ, or in vitro.

In a preferred embodiment, we describe a process for the simultaneous or coordinated delivery of an siRNA(s) together with a small molecule drug to a cell or tissue, i.e. combination therapy. The siRNA is delivered to the cell or tissue to exert an effect on the levels of a protein, such as an enzyme, in the cell or tissue. The siRNA-induced reduction in the amount the protein can enhance or alter the effect of a small molecule drug. In a preferred embodiment, a lower dose of the small molecule is required to generate a specific cellular outcome when combined with siRNA delivery. By using siRNA to reduce the amount of a target protein, the dose of drug required to inhibit an endogenous cellular protein is lowered or its efficacy is increased. The drug and the siRNA may both affect the same gene/gene product. Alternatively, the siRNA and drug may be chosen to work cooperatively through inhibition of different genes.

In a preferred embodiment, an inhibitor may be delivered to a cell in a mammal for the purposes of inhibiting a target gene to provide a therapeutic effect. The target gene is selected from the group that comprises: dysfunctional endogenous genes and viral or other infectious agent genes. Dysfunctional endogenous genes include dominant genes which cause disease and cancer genes.

In a preferred embodiment, an inhibitor is delivered to a mammalian cell in vivo for the treatment of a disease or infection. The inhibitor reduces expression of a viral or bacterial gene. The inhibitor may reduce or block microbe production, virulence, or both. Delivery of the inhibitor may delay progression of disease until endogenous immune protection can be acquired. In a preferred embodiment, combinations of effective inhibitors or combinations of inhibitor and small molecule drugs targeted to the same or different viral genes or classes of genes (e.g., transcription, replication, virulence, etc) are delivered to an infected mammalian cell in vivo. Alternatively, instead of inhibiting an infectious agent gene, the inhibitor may decrease expression of an endogenous host gene to reduce virulence of the pathogen. The inhibitor may be delivered to a cell in a mammal to reduce expression of a cellular receptor.

In a preferred embodiment, an inhibitor is delivered to a mammalian cell in vivo to modulate immune response. Since host immune response is responsible for the toxicity of some infectious agents, reducing this response may increase the survival of an infected mammal. Also, inhibition of immune response is beneficial for a number of other therapeutic purposes, including gene therapy, where immune reaction often greatly limits transgene expression, organ transplantation, and autoimmune disorders.

In a preferred embodiment, an inhibitor is delivered to a mammalian cell for the purpose of facilitating pharmaceutical drug discovery or target validation. The mammalian cell may be in vitro or in vivo. Specific inhibition of a target gene can aid in determining whether an inhibition of a protein or gene has a significant phenotypic effect. Specific inhibition of a target gene can also be used to study the target gene's effect on the cell.

Further objects, features, and advantages of the invention will be apparent from the following detailed description when taken in conjunction with the accompanying drawings.

### BRIEF DESCRIPTION OF FIGURES

FIG. 1. siRNA is efficiently delivered to multiple tissue types in mice in vivo and the delivered siRNA is highly effective for inhibiting target gene expression in all organs tested.

FIG. 2. Intravascular delivery of siRNA inhibits EGFP expression in the liver of transgenic mice. EGFP (green), phalloidin (red). 10 week old mice (strain C57BL/6-TgN (ACTbEGFP) 10sb) expressing EGFP were injected with 50 μg siRNA (mice #1 and 2), 50 μg control siRNA (mice #3 and 4) or were not injected (mouse #5). Livers were harvested 30 h post-injection, sectioned, fixed, and counterstained with Alexa 568 phalloidin in order to visualize cell outlines. Images were acquired using a Zeiss Axioplan fluorescence microscope outfitted with a Zeiss AxioCam digital camera.

FIG. 3. A) Delivery of siRNA-Luc+. Maximal inhibition is achieved at 10 nM siRNA-Luc+. B) Delivery of morpholino-Luc+. Maximal specfic inhibition is achieved at 100 nM morpholino-Luc+. C) Comparison of inhibitory power of siRNA-Luc+ (1.0 nM) alone, morpholino-Luc+ (100 nM) alone and siRNA-Luc+ (1.0 nM) plus morpholino (100 nM) together. When siRNA and morpholino are added together at these concentrations, the degree of inhibition is greater than either siRNA or morphlino alone. D) Comparison of inhibitory power of siRNA-Luc+(10 nM) alone, morpholino-Luc+ (100 nM) alone and siRNA-Luc+ (10 nM) plus morpholino (100 nM) together. When siRNA and morpholino are added together at these concentrations, the degree of inhibition is greater than either siRNA or morphlino alone.

FIG. 4. Peak gene transfer activities of DNA/brPEI/polyanion complexes applied to HUH7 cells in 100% bovine serum. The peak activities were obtained in titration experiments. pAA, polyacrylic acid; pAsp, polyaspartic acid; pGlu, polyglutamic acid; SPLL, succinylated poly-L-lysine.

FIG. 5. Graph illustrating reduction in PPAR levels following delivery of PPAR-siRNA expression cassettes in vivo.

FIG. 6A-6B. A. Graph illustrating levels of HMG-CoA reductase mRNA in mice treated with 50 mg/kg atorvastatin. B. Graph illustrating prevention of atorvastatin-induced upregulation of HMGCR levels in vitro by co-delivery of HMGCR siRNA.

FIG. 7. Relative levels of PPARα mRNA in groups of mice injected with siRNAs. mRNA levels are shown relative to total input RNA. Black bar=Experimental group; Grey bars=control group.

FIG. 8A-8C. A. Graph illustrating effect of statin treatment on LDLR mRNA in primary hepatocytes. B. Graph illustrating relative levels of LDLR mRNA in hepatocytes treated with statins and siRNAs. Dark bars=HMGCR siRNA-treated cells; Light bars=GL3-treated cells. C. Graph illustrating lower doses of atorvastatin necessary to get comparable statin/no statin ratios in cells treated with HMGCR siRNAs.

### DETAILED DESCRIPTION

We have found that an intravascular route of administration allows a polynucleotide-based expression inhibitor (inhibitor) to be delivered to a mammalian cell in a more even distribution than direct parenchymal injections. The efficiency of inhibitor delivery may be increased by increasing the permeability of the tissue's blood vessel. Permeability is increased by increasing the intravascular hydrostatic pressure (above, for example, the resting diastolic blood pressure in a blood vessel), delivering the injection fluid rapidly (injecting the injection fluid rapidly), using a large injection volume, and/or increasing permeability of the vessel wall.

A polynucleotide-based gene expression inhibitor comprises any polynucleotide containing a sequence whose presence or expression in a cell causes the degradation of or inhibits the function, transcription, or translation of a gene in a sequence-specific manner. Polynucleotide-based expression inhibitors may be selected from the group comprising: siRNA, microRNA, interfering RNA or RNAi, dsRNA, ribozymes, antisense polynucleotides, and DNA expression cassettes encoding siRNA, microRNA, dsRNA, ribozymes or antisense nucleic acids. SiRNA comprises a double stranded structure typically containing 15-50 base pairs and preferably 19-25 base pairs and having a nucleotide sequence identical or nearly identical to an expressed target gene or RNA within the cell. An siRNA may be composed of two annealed polynucleotides or a single polynucleotide that forms a hairpin structure. MicroRNAs (mRNAs) are small noncoding polynucleotides, about 22 nucleotides long, that direct destruction or translational repression of their mRNA targets. Antisense polynucleotides comprise sequence that is complimentary to a gene or mRNA. Antisense polynucleotides include, but are not limited to: morpholinos, 2′-O-methyl polynucleotides, DNA, RNA and the like. The polynucleotide-based expression inhibitor may be polymerized in vitro, recombinant, contain chimeric sequences, or derivatives of these groups. The polynucleotide-based expression inhibitor may contain ribonucleotides, deoxyribonucleotides, synthetic nucleotides, or any suitable combination such that the target RNA and/or gene is inhibited.

A delivered inhibitor can stay within the cytoplasm or nucleus. The inhibitor can be delivered to a cell to inhibit expression of an endogenous or exogenous nucleotide sequence or to affect a specific physiological characteristic not naturally associated with the cell.

An inhibitor can be delivered to a cell in order to produce a cellular change that is therapeutic. The inhibitor can be delivered either directly to the organism in situ or indirectly by transfer to a cell ex vivo that is then transplanted into the organism. Entry into the cell is required for the inhibitor to block the production of a protein or to decrease the amount of a target RNA. Diseases, such as autosomal dominant muscular dystrophies, which are caused by dominant mutant genes, are examples of candidates for treatment with therapeutic inhibitors such as siRNA. Delivery of the inhibitor would block production of the dominant protein without affecting the normal protein thereby lessening the disease.

We demonstrate that delivery of siRNA and antisense inhibitors to cells of post-embryonic mice and rats interferes with specific gene expression in those cells. The inhibition is gene specific and does not cause general translational arrest. Thus RNAi can be effective in post-embryonic mammalian cells in vivo.

Many disease treatments aim to inhibit the activity of a well-defined protein to give a therapeutic effect. Such effects are realized only when the levels of active target protein drop below a certain threshold. SiRNA may be used to reduce the amount of target protein to be inhibited by small molecule drugs. This reduction in protein levels results in a lower dosage of the small molecule drug be necessary to gain a clinical outcome, perhaps leading to significantly lower recommended doses and reduced side effects. This strategy may help lower the hurdles to successful treatments for a variety of diseases. In addition, it may facilitate drug discovery and research by providing a method of sensitizing cells to the action of a small molecule targeting a particular gene product.

Combination therapy is defined as the simultaneous administration of multiple treatments to treat a single pathogenic or disease state. This strategy has been used successfully to treat a variety of diseases. For example, chemotherapy and radiation remain a common treatment of nearly all cancers. Furthermore, many of the newer anti-cancer drugs are measured for efficacy in combination with traditional therapies like chemotherapy and radiation. In addition, HIV combination therapy and its cocktail of protease inhibitors and reverse transcriptase inhibitors has returned a sort of normalcy to the lives of many AIDS patients.

In a preferred embodiment, an siRNA (siRNA-HMGCR) directed against the gene 3-alpha-hydroxy-3-methylglutaryl-CoA reductase (HMG CoA reductase; HMGCR) is delivered to cells. Under effective delivery conditions, siRNA-HMGCR affects HMGCR enzyme levels. In a preferred embodiment, siRNA-HMGCR influences lipid homeostasis in a mammal. This effect can be used to study lipid biochemistry and metabolism in cells in vitro and in vivo (e.g., for the purpose of target validation). In another application, this effect can be used for therapeutic purposes. In another preferred embodiment, siRNAs directed against other genes known to be involved in the lipid metabolism are delivered to cells. In another preferred embodiment, siRNAs directed against other genes are delivered to cells.

The term nucleic acid, or polynucleotide, is a term of art that refers to a string of at least two nucleotides. Nucleotides are the monomeric units of nucleic acid polymers. Polynucleotides with less than 120 monomeric units are often called oligonucleotides. Natural nucleic acids have a deoxyribose- or ribose-phosphate backbone while artificial polynucleotides are polymerized in vitro and contain the same or similar bases but may contain other types of backbones. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups on the base such as, but not limited to, amines, alcohols, thiols, carboxylates, and alkylhalides. The term base encompasses any of the known base analogs of DNA and RNA. The term includes deoxyribonucleic acid (DNA) and ribonucleic acid (RNA). DNA may be in form of cDNA, in vitro polymerized DNA, plasmid DNA, parts of a plasmid DNA, genetic material derived from a virus, linear DNA, chromosomal DNA, an oligonucleotide, antisense DNA, or derivatives of these groups. RNA may be in the form of tRNA (transfer RNA), snRNA (small nuclear RNA), rRNA (ribosomal RNA), mRNA (messenger RNA), antisense RNA, siRNA (small interfering RNA), dsRNA (double stranded RNA), RNAi, ribozymes, in vitro polymerized RNA, or derivatives of these groups.

The term deliver means that the inhibitor becomes associated with the cell thereby altering the properties of the cell by inhibiting function of an RNA. The inhibitor can be on the membrane of the cell or inside the cytoplasm, nucleus, or other organelle of the cell. Other terms sometimes used interchangeably with deliver include transfect, transfer, or transform. In vivo delivery of an inhibitor means to transfer the inhibitor from a container outside a mammal to near or within the outer cell membrane of a cell in the mammal. The inhibitor can interfere with RNA function in either the nucleus or cytoplasm.

Using the described invention, inhibitors are efficiently delivered to cells in culture, i.e., in vitro. These include a number of cell lines that can be obtained from American Type Culture Collection (Bethesda) such as, but not limited to: 3T3 (mouse fibroblast) cells, Rat1 (rat fibroblast) cells, CHO (Chinese hamster ovary) cells, CV-1 (monkey kidney) cells, COS (monkey kidney) cells, 293 (human embryonic kidney) cells, HeLa (human cervical carcinoma) cells, HepG2 (human hepatocytes) cells, Sf9 (insect ovarian epithelial) cells and the like.

The invention also describes the delivery of an inhibitor to a cell that is in vivo, in situ, ex vivo or a primary cell. Primary cells include, but are not limited to, primary liver cells and primary muscle cells and the like. For primary cells, the cells within the tissue are separated by mincing and digestion with enzymes such as trypsin or collagenases which destroy the extracellular matrix. Tissues consist of several different cell types. Purification methods such as gradient centrifugation or antibody sorting can be used to obtain purified amounts of the preferred cell type. For example, primary myoblasts are separated from contaminating fibroblasts using Percoll (Sigma) gradient centrifugation.

Parenchymal cells are the distinguishing cells of a gland or organ contained in and supported by the connective tissue framework. The parenchymal cells typically perform a function that is unique to the particular organ. The term “parenchymal” often excludes cells that are common to many organs and tissues such as fibroblasts and endothelial cells within blood vessels.

For example, in a liver organ, the parenchymal cells include hepatocytes, Kupffer cells and the epithelial cells that line the biliary tract and bile ductules. The major constituent of the liver parenchyma are polyhedral hepatocytes (also known as hepatic cells) that presents at least one side to an hepatic sinusoid and opposed sides to a bile canaliculus. Liver cells that are not parenchymal cells include cells within the blood vessels such as the endothelial cells or fibroblast cells. In one preferred embodiment hepatocytes are targeted by injecting the inhibitor or inhibitor complex into the portal vein or bile duct of a mammal.

In striated muscle, the parenchymal cells include myoblasts, satellite cells, myotubules, and myofibers. In cardiac muscle, the parenchymal cells include the myocardium also known as cardiac muscle fibers or cardiac muscle cells and the cells of the impulse connecting system such as those that constitute the sinoatrial node, atrioventricular node, and atrioventricular bundle.

Vessels comprise internal hollow tubular structures connected to a tissue or organ within the body. Bodily fluid flows to or from the body part within the cavity of the tubular structure. Examples of bodily fluid include blood, lymphatic fluid, or bile. Examples of vessels include arteries, arterioles, capillaries, venules, sinusoids, veins, lymphatics, and bile ducts. Afferent blood vessels of organs are defined as vessels which are directed towards the organ or tissue and in which blood flows towards the organ or tissue under normal physiological conditions. Conversely, efferent blood vessels of organs are defined as vessels which are directed away from the organ or tissue and in which blood flows away from the organ or tissue under normal physiological conditions. In the liver, the hepatic vein is an efferent blood vessel since it normally carries blood away from the liver into the inferior vena cava. Also in the liver, the portal vein and hepatic arteries are afferent blood vessels in relation to the liver since they normally carry blood towards the liver. Insertion of the inhibitor or inhibitor complex into a vessel enables the inhibitor to be delivered to parenchymal cells more efficiently and in a more even distribution compared with direct parenchymal injections.

In a preferred embodiment, the permeability of the vessel is increased. Efficiency of inhibitor delivery is increased by increasing the permeability of a vessel within the target tissue. Permeability is defined here as the propensity for macromolecules such as an inhibitor to exit the vessel and enter extravascular space. One measure of permeability is the rate at which macromolecules move out of the vessel. Another measure of permeability is the lack of force that resists the movement of inhibitors being delivered to leave the intravascular space.

Rapid injection may be combined with obstructing the outflow to increase permeability. To obstruct, in this specification, is to block or inhibit inflow or outflow of fluid through a vessel. For example, an afferent vessel supplying an organ is rapidly injected and the efferent vessel draining the tissue is ligated transiently. The efferent vessel (also called the venous outflow or tract) draining outflow from the tissue is also partially or totally clamped for a period of time sufficient to allow delivery of a polynucleotide. In the reverse, an efferent is injected and an afferent vessel is occluded.

In another preferred embodiment, the pressure of a vessel is increased by increasing the osmotic pressure within the vessel. Typically, hypertonic solutions containing salts such as NaCl, sugars or polyols such as mannitol are used. Hypertonic means that the osmolarity of the injection solution is greater than physiological osmolarity. Isotonic means that the osmolarity of the injection solution is the same as the physiological osmolarity (the tonicity or osmotic pressure of the solution is similar to that of blood). Hypertonic solutions have increased tonicity and osmotic pressure relative to the osmotic pressure of blood and cause cells to shrink.

In another preferred embodiment, the permeability of a vessel can be increased by a biologically-active molecule. A biologically-active molecule is a protein or a simple chemical such as papaverine or histamine that increases the permeability of the vessel by causing a change in function, activity, or shape of cells within the vessel wall such as the endothelial or smooth muscle cells. Typically, biologically-active molecules interact with a specific receptor or enzyme or protein within the vascular cell to change the vessel's permeability. Biologically-active molecules include vascular permeability factor (VPF) which is also known as vascular endothelial growth factor (VEGF). Another type of biologically-active molecule can increase permeability by changing the extracellular connective material. For example, an enzyme could digest the extracellular material and increase the number and size of the holes of the connective material.

In a preferred embodiment, an inhibitor or inhibitor-containing complex is injected into a vessel in a large injection volume. The injection volume is dependent on the size of the animal to be injected and can be from 1.0 to 3.0 ml or greater for small animals (i.e. tail vein injections into mice). The injection volume for rats can be from 6 to 35 ml or greater. The injection volume for primates can be 70 to 200 ml or greater. The injection volumes in terms of ml/body weight can be 0.03 ml/g to 0.1 ml/g or greater.

The injection volume can also be related to the target tissue. For example, delivery of a non-viral vector with an inhibitor to a limb can be aided by injecting a volume greater than 5 ml per rat limb or greater than 70 ml for a primate. The injection volumes in terms of ml/limb muscle are usually within the range of 0.6 to 1.8 ml/g of muscle but can be greater. In another example, delivery of an inhibitor or inhibitor complex to liver in mice can be aided by injecting the inhibitor in an injection volume from 0.6 to 1.8 ml/g of liver or greater. In another example delivering an inhibitor to a limb of a primate (rhesus monkey), the inhibitor or complex can be in an injection volume from 0.6 to 1.8 ml/g of limb muscle or anywhere within this range.

In another embodiment the injection fluid is injected into a vessel rapidly. The speed of the injection is partially dependent on the volume to be injected, the size of the vessel into which the volume is injected, and the size of the animal. In one embodiment the total injection volume (1-3 ml) can be injected from 15 to 5 seconds into the vascular system of mice. In another embodiment the total injection volume (6-35 ml) can be injected into the vascular system of rats from 20 to 7 seconds. In another embodiment the total injection volume (80-200 ml) can be injected into the vascular system of monkeys from 120 seconds or less.

In another embodiment a large injection volume is used and the rate of injection is varied. Injection rates of less than 0.012 ml per gram (animal weight) per second are used in this embodiment. In another embodiment injection rates of less than 0.2 ml per gram (target tissue weight) per second are used for gene delivery to target organs. In another embodiment injection rates of less than 0.06 ml per gram (target tissue weight) per second are used for gene delivery into limb muscle and other muscles of primates.

Polymers have been used in research for the delivery of nucleic acids to cells. One of the several methods of nucleic acid delivery to the cells is the use of nucleic acid/polycation complexes. It has been shown that cationic proteins, like histones and protamines, or synthetic polymers, like polylysine, polyarginine, polyornithine, DEAE dextran, polybrene, and polyethylenimine, but not small polycations like spermine may be effective intracellular DNA delivery agents. Multivalent cations with a charge of three or higher have been shown to condense nucleic acid when 90% or more of the charges along the sugar-phosphate backbone are neutralized. The volume which one polynucleotide molecule occupies in a complex with polycations is lower than the volume of a free polynucleotide molecule. Polycations also provide attachment of polynucleotide to a cell surface. The polymer forms a cross-bridge between the polyanionic nucleic acid and the polyanionic surface of the cell. As a result, the mechanism of nucleic acid translocation to the intracellular space might be non-specific adsorptive endocytosis. Furthermore, polycations provide a convenient linker for attaching specific ligands to the complex. The nucleic acid/polycation complexes could then be targeted to specific cell types. Complex formation also protects against nucleic acid degradation by nucleases present in serum as well as in endosomes and lysosomes. Protection from degradation in endosomes/lysosomes is enhanced by preventing organelle acidification. Disruption of endosomal/lysosomal function may also be accomplished by linking endosomal or membrane disruptive agents to the polycation or complex.

A DNA-binding protein is a protein that associates with nucleic acid under conditions described in this application and forms a complex with nucleic acid with a high binding constant. The DNA-binding protein can be used in an effective amount in its natural form or a modified form for this process. An “effective amount” of the polycation is an amount that will allow delivery of the inhibitor to occur.

A non-viral vector is defined as a vector that is not assembled within an eukaryotic cell including non-viral inhibitor/polymer complexes, inhibitor with transfection enhancing compounds and inhibitor+amphipathic compounds.

A molecule is modified, to form a modification through a process called modification, by a second molecule if the two become bonded through a covalent bond. That is, the two molecules form a covalent bond between an atom from one molecule and an atom from the second molecule resulting in the formation of a new single molecule. A chemical covalent bond is an interaction, bond, between two atoms in which there is a sharing of electron density. Modification also means an interaction between two molecules through a noncovalent bond. For example crown ethers can form noncovalent bonds with certain amine groups.

Functional groups include cell targeting signals, nuclear localization signals, compounds that enhance release of contents from endosomes or other intracellular vesicles (releasing signals), and other compounds that alter the behavior or interactions of the compound are complex to which they are attached.

Cell targeting signals are any signals that enhance the association of the biologically active compound with a cell. These signals can modify a biologically active compound such as drug or nucleic acid and can direct it to a cell location (such as tissue) or location in a cell (such as the nucleus) either in culture or in a whole organism. The signal may increase binding of the compound to the cell surface and/or its association with an intracellular compartment. By modifying the cellular or tissue location of the foreign gene, the function of the biologically active compound can be enhanced. The cell targeting signal can be, but is not limited to, a protein, peptide, lipid, steroid, sugar, carbohydrate, (non-expressing) polynucleic acid or synthetic compound. Cell targeting signals such as ligands enhance cellular binding to receptors. A variety of ligands have been used to target drugs and genes to cells and to specific cellular receptors. The ligand may seek a target within the cell membrane, on the cell membrane or near a cell. Binding of ligands to receptors typically initiates endocytosis. Ligands include agents that target to the asialoglycoprotein receptor by using asialoglycoproteins or galactose residues. Other proteins such as insulin, EGF, or transferrin can be used for targeting. Peptides that include the RGD sequence can be used to target many cells. Chemical groups that react with thiol, sulfhydryl, or disulfide groups on cells can also be used to target many types of cells. Folate and other vitamins can also be used for targeting. Other targeting groups include molecules that interact with membranes such as lipids, fatty acids, cholesterol, dansyl compounds, and amphotericin derivatives. In addition viral proteins could be used to bind cells.

Transfection—The process of delivering a polynucleotide to a cell has been commonly termed transfection or the process of transfecting and also it has been termed transformation. The term transfecting as used herein refers to the introduction of a polynucleotide or other biologically active compound into cells. The polynucleotide may be delivered to the cell for research purposes or to produce a change in a cell that can be therapeutic. The delivery of a polynucleotide for therapeutic purposes is commonly called gene therapy. Gene therapy is the purposeful delivery of genetic material to somatic cells for the purpose of treating disease or biomedical investigation. The delivery of a polynucleotide can lead to modification of the genetic material present in the target cell.

Transfection agent—A transfection reagent or delivery vehicle is a compound or compounds that bind(s) to or complex(es) with oligonucleotides and polynucleotides, and mediates their entry into cells. Examples of transfection reagents include, but are not limited to, cationic liposomes and lipids, polyamines, calcium phosphate precipitates, histone proteins, polyethylenimine, polylysine, and polyampholyte complexes. It has been shown that cationic proteins like histones and protamines, or synthetic polymers like polylysine, polyarginine, polyomithine, DEAE dextran, polybrene, and polyethylenimine may be effective intracellular delivery agents. Typically, the transfection reagent has a component with a net positive charge that binds to the oligonucleotide's or polynucleotide's negative charge.

Biologically active compound—A biologically active compound is a compound having the potential to react with biological components. More particularly, biologically active compounds utilized in this specification are designed to change the natural processes associated with a living cell. For purposes of this specification, a cellular natural process is a process that is associated with a cell before delivery of a biologically active compound. Biologically active compounds may be selected from the group comprising: pharmaceuticals, drugs, proteins, peptides, polypeptides, hormones, cytokines, antigens, viruses, oligonucleotides, and nucleic acids.

We have disclosed gene expression and/or inhibition achieved from reporter genes in specific tissues. Levels of a gene product, including reporter (marker) gene products, are measured which then indicate a reasonable expectation of similar amounts of gene expression by delivering other polynucleotides. Levels of treatment considered beneficial by a person having ordinary skill in the art differ from disease to disease, for example: Hemophilia A and B are caused by deficiencies of the X-linked clotting factors VIII and IX, respectively. Their clinical course is greatly influenced by the percentage of normal serum levels of factor VIII or IX: <2%, severe; 2-5%, moderate; and 5-30% mild. Thus, an increase from 1% to 2% of the normal level of circulating factor in severe patients can be considered beneficial. Levels greater than 6% prevent spontaneous bleeds but not those secondary to surgery or injury. A person having ordinary skill in the art of gene therapy would reasonably anticipate beneficial levels of expression of a gene specific for a disease based upon sufficient levels of marker gene results. In the hemophilia example, if marker genes were expressed to yield a protein at a level comparable in volume to 2% of the normal level of factor VIII, it can be reasonably expected that the gene coding for factor VIII would also be expressed at similar levels. Thus, reporter or marker genes such as the genes for luciferase and β-galactosidase serve as useful paradigms for expression of intracellular proteins in general. Similarly, reporter or marker genes, such as the gene for secreted alkaline phosphatase (SEAP), serve as useful paradigms for secreted proteins in general.

### EXAMPLES

The following examples are intended to illustrate, but not limit, the present invention.

### Example 1

Inhibition of luciferase gene expression by siRNA in liver cells in vivo. Single-stranded, gene-specific sense and antisense RNA oligomers with overhanging 3′ deoxyribonucleotides were prepared and purified by PAGE. The two oligomers, 40 μM each, were annealed in 250 μl buffer containing 50 mM Tris-HCl, pH 8.0 and 100 mM NaCl, by heating to 94° C. for 2 minutes, cooling to 90° C. for 1 minute, then cooling to 20° C. at a rate of 1° C. per minute. The resulting siRNA was stored at −20° C. prior to use.

The sense oligomer with identity to the luc+ gene has the sequence: 5′-rCrUrUrArCrGrC-rUrGrArGrUrArCrUrUrCrGrATT-3′ (SEQ ID 4), which corresponds to positions 155-173 of the luc+ reading frame. The letter “r” preceding a nucleotide indicates that nucleotide is a ribonucleotide. The antisense oligomer with identity to the luc+ gene has the sequence: 5′-rUrCrGrArArGrUrArCrUrCrArGrCrGrUrArArGTT-3′ (SEQ ID 5), which corresponds to positions 155-173 of the luc+ reading frame in the antisense direction. The letter “r” preceding a nucleotide indicates that nucleotide is a ribonucleotide. The annealed oligomers containing luc+ coding sequence are referred to as siRNA-luc+.

The sense oligomer with identity to the ColE1 replication origin of bacterial plasmids has the sequence: 5′-rGrCrGrArUrArArGrUrCrGrUrGrUrCrUrUrArCTT-3′ (SEQ ID 6). The letter “r” preceding a nucleotide indicates that nucleotide is a ribonucleotide. The antisense oligomer with identity to the ColE1 origin of bacterial plasmids has the sequence: 5′-rGrUrArArGrArCrArCrGrArCrUrUrArUrCrGrCTT-3′ (SEQ ID 7). The letter “r” preceding a nucleotide indicates that nucleotide is a ribonucleotide. The annealed oligomers containing ColE1 sequence are referred to as siRNA-ori.

Plasmid pMIR48 (10 μg), containing the luc+ coding region (Promega Corp.) and a chimeric intron downstream of the cytomegalovirus major immediate-early enhancer/promoter, was mixed with 0.5 or 5 μg siRNA-luc+, diluted in 1-3 ml Ringer's solution (147 mM NaCl, 4 mM KCl, 1.13 mM CaCl₂) and injected into the tail vein of ICR mice over 7-120 seconds. One day after injection, the livers were harvested and homogenized in lysis buffer (0.1% Triton X-100, 0.1 M K-phosphate, 1 mM DTT, pH 7.8). Insoluble material was cleared by centrifugation. 10 μl of the cellular extract or extract diluted 10× was analyzed for luciferase activity using the Enhanced Luciferase Assay kit (Mirus).

Co-injection of 10 μg pMIR48 and 0.5 μg siRNA-luc+ results in 69% inhibition of Luc+ activity as compared to injection of 10 μg pMIR48 alone. Co-injection of 5 μg siRNA-luc+ with 10 μg pMIR48 results in 93% inhibition of Luc+ activity.

### Example 2

Inhibition of Luciferase expression by siRNA is gene specific in liver in vivo. Two plasmids were injected simultaneously either with or without siRNA-luc+ as described in Example 1. The first plasmid, pGL3 control (Promega Corp, Madison, Wis.), contains the luc+ coding region and a chimeric intron under transcriptional control of the simian virus 40 enhancer and early promoter region. The second, pRL-SV40, contains the coding region for the Renilla reniformis luciferase under transcriptional control of the Simian virus 40 enhancer and early promoter region.

10 μg pGL3 control and 1 μg pRL-SV40 was injected as described in Example 1 with 0, 0.5 or 5.0 μg siRNA-luc+. One day after injection, the livers were harvested and homogenized as described in Example 1. Luc+ and Renilla Luc activities were assayed using the Dual Luciferase Reporter Assay System (Promega). Ratios of Luc+ to Renilla Luc were normalized to the no siRNA-Luc+ control. siRNA-luc+ specifically inhibited the target Luc+ expression 73% at 0.5 μg co-injected siRNA-luc+ and 82% at 5.0 μg co-injected siRNA-luc+.

### Example 3

Inhibition of Luciferase expression by siRNA is gene specific and siRNA specific in liver in vivo. 10 μg pGL3 control and 1 μg pRL-SV40 were injected as described in Example 1 with either 5.0 μg siRNA-luc+ or 5.0 control siRNA-ori. One day after injection, the livers were harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in liver by 93% compared to siRNA-ori indicating inhibition by siRNAs is sequence specific in this organ.

### Example 4

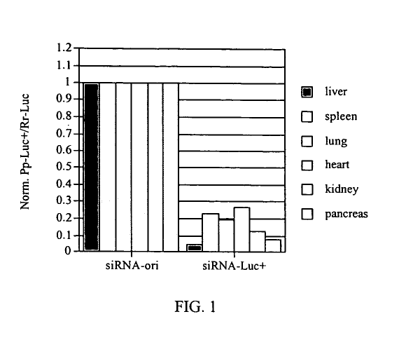

In vivo delivery of siRNA by increased-pressure intravascular injection results in strong inhibition of target gene expression in a variety of organs. 10 μg pGL3 Control and 1 μg pRL-SV40 were co-injected with 5 μg siRNA-Luc+ or 5 μg control siRNA (siRNA-ori) targeted to sequence in the plasmid backbone as in example 1. One day after injection, organs were harvested and homogenized and the extracts assayed for target firefly luciferase+ activity and control Renilla luciferase activity. Firefly luciferase+activity was normalized to that Renilla luciferase activity in order to compensate for differences in transfection efficiency between animals. Results are shown in FIG. 1. Expression of firefly luciferase+ activity was strongly inhibited in liver (95% inhibition), spleen (77%), lung (81%), heart (74%), kidney (87%) and pancreas (92%), compared to animals injected with the control siRNA-ori. Animals injected with plasmid alone contained similar luciferase activities to those injected with the control siRNA-ori alone, indicating that the presence of siRNA alone does not significantly affect in vivo plasmid DNA transfection efficiencies (data not shown).

These results (FIG. 1) indicate effective delivery of siRNA to a number of different tissue types in vivo. Furthermore, the fact that expression of the control Renilla luciferase was not affected by the presence of siRNA suggests that siRNA is not inducing an interferon response. This is the first demonstration of the effectiveness of siRNA for inhibiting gene expression in post-embryonic mammalian tissues and demonstrates siRNA could be delivered to these organs to inhibit gene expression.

### Example 5

Inhibition of Luciferase expression by siRNA is gene specific and siRNA specific in liver after bile duct delivery in vivo. 10 μg pGL3 control and 1 μg pRL-SV40 with 5.0 μg siRNA-luc+ or 5.0 siRNA-ori were injected into the bile duct of mice. A total volume of 1 ml in Ringer's buffer was delivered at 6 ml/min. The inferior vena cava was clamped above and below the liver before injection and clamps were left on for two minutes after injection. One day after injection, the liver was harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in liver by 88% compared to the control siRNA-ori.

### Example 6

Inhibition of Luciferase expression by siRNA is gene specific and siRNA specific in muscle in vivo after arterial delivery. 10 μg pGL3 control and 1 μg pRL-SV40 with 5.0 μg siRNA-luc+ or 5.0 siRNA-ori were injected into iliac artery of rats under increased pressure. Specifically, animals were anesthetized and the surgical field shaved and prepped with an antiseptic. The animals were placed on a heating pad to prevent loss of body heat during the surgical procedure. A midline abdominal incision will be made after which skin flaps were folded away and held with clamps to expose the target area. A moist gauze was applied to prevent excessive drying of internal organs. Intestines were moved to visualize the iliac veins and arteries. Microvessel clips were placed on the external iliac, caudal epigastric, internal iliac, deferent duct, and gluteal arteries and veins to block both outflow and inflow of the blood to the leg. An efflux enhancer solution (e.g., 0.5 mg papaverine in 3 ml saline) was injected into the external iliac artery though a 25 g needle, followed by the plasmid DNA and siRNA containing solution (in 10 ml saline) 1-10 minutes later. The solution was injected in approximately 10 seconds. The microvessel clips were removed 2 minutes after the injection and bleeding was controlled with pressure and gel foam. The abdominal muscles and skin were closed with 4-0 dexon suture.

Four days after injection, rats were sacrificed and the quadriceps and gastrocnemius muscles were harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in quadriceps and gastrocnemius by 85% and 92%, respectively, compared to the control siRNA-ori.

### Example 7

RNAi of SEAP reporter gene expression using siRNA in vivo. Single-stranded, SEAP-specific sense and antisense RNA oligomers with overhanging 3′ deoxyribonucleotides were prepared and purified by PAGE. The two oligomers, 40 μM each, were annealed in 250 μl buffer containing 50 mM Tris-HCl, pH 8.0 and 100 mM NaCl, by heating to 94° C. for 2 min, cooling to 90° C. for 1 min, then cooling to 20° C. at a rate of 1° C. per min. The resulting siRNA was stored at −20° C. prior to use.

The sense oligomer with identity to the SEAP reporter gene has the sequence: 5′-rArGrGrG-rCrArArCrUrUrCrCrArGrArCrCrArUTT-3′ (SEQ ID 8), which corresponds to positions 362-380 of the SEAP reading frame in the sense direction. The letter “r” preceding a nucleotide indicates that nucleotide is a ribonucleotide. The antisense oligomer with identity to the SEAP reporter gene has the sequence: 5′-rArUrGrGrUrCrUrGrGrArArGrUrUrG-rCrCrCrUTT-3′ (SEQ ID 9), which corresponds to positions 362-380 of the SEAP reading frame in the antisense direction. The letter “r” preceding a nucleotide indicates that nucleotide is a ribonucleotide. The annealed oligomers containing SEAP coding sequence are referred to as siRNA-SEAP.

Plasmid pMIR141 (10 μg), containing the SEAP coding region under transcriptional control of the human ubiquitin C promoter and the human hepatic control region of the apolipoprotein E gene cluster, was mixed with 0.5 or 5 μg siRNA-SEAP or 5 μg siRNA-ori, diluted in 1-3 ml Ringer's solution (147 mM NaCl, 4 mM KCl, 1.13 mM CaCl₂), and injected into the tail vein over 7-120 seconds. Control mice also included those injected with pMIR141 alone. Each mouse was bled from the retro-orbital sinus one day after injection. Cells and clotting factors were pelleted from the blood to obtain serum. The serum was then evaluated for the presence of SEAP by a chemiluminescence assay using the Tropix Phospha-Light kit. Results showed that SEAP expression was inhibited by 59% when 0.5 μg siRNA-SEAP was delivered and 83% when 5.0 μg siRNA-SEAP was delivered. No decrease in SEAP expression was observed when 5.0 μg siRNA-ori was delivered indicating the decrease in SEAP expression by siRNA-SEAP was gene specific.

TABLE 1
Inhibition of SEAP expression in vivo following delivery
by tail vain injection of SEAP expression
plasmid and siRNA-SEAP.

| injection | Ave. SEAP (ng/ml) | St. Dev. |
|---|---|---|
| plasmid only | 2239 | 1400 |
| siRNA-ori (5.0 μg) | 2897 | 1384 |
| siRNA-SEAP (0.5 μg) | 918 | 650 |
| siRNA-SEAP (5.0 μg) | 384 | 160 |

### Example 8

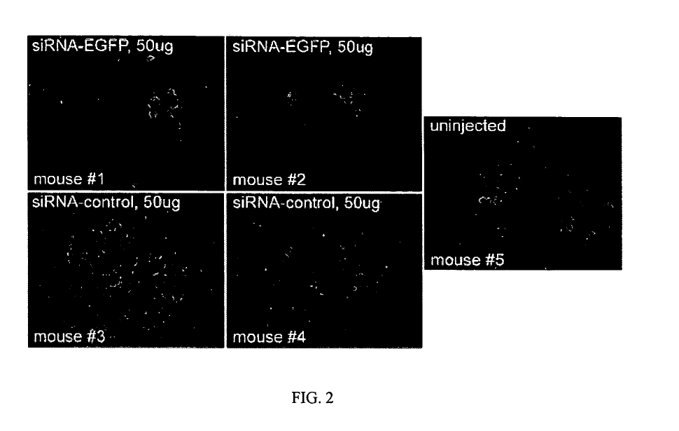

Inhibition of green fluorescent protein in transgenic mice using siRNA. The commercially available mouse strain C57BL/6-TgN(ACThEGFP)10sb (The Jackson Laboratory) has been reported to express enhanced green fluorescent protein (EGFP) in all cell types except erythrocytes and hair. These mice were injected with siRNA targeted against EGFP (siRNA-EGFP) or a control siRNA (siRNA-control) using the increased pressure tail vein intravascular injection method described previously. 30 h post-injection, the animals were sacrificed and sections of the liver were prepared for fluorescence microscopy. Liver sections from animals injected with 50 μg siRNA-EGFP displayed a substantial decrease in the number of cells expressing EGFP compared to animals injected with siRNA-control or mock injected (FIG. 2). The data shown here demonstrate effective delivery of siRNA-EGFP to the liver. The delivered siRNA-EGFP then inhibited EGFP gene expression in the mice. We have therefore shown the ability of siRNA to inhibit the expression of an endogenous gene product in post-natal mammals.

### Example 9

Inhibition of endogenous mouse cytosolic alanine aminotransferase (ALT) expression after in vivo delivery of siRNA. Single-stranded, cytosolic alanine aminotransferase-specific sense and antisense RNA oligomers with overhanging 3′ deoxyribonucleotides were prepared and purified by PAGE. The two oligomers, 40 μM each, were annealed in 250 μl buffer containing 50 mM Tris-HCl, pH 8.0 and 100 mM NaCl, by heating to 94° C. for 2 minutes, cooling to 90° C. for 1 minute, then cooling to 20° C. at a rate of 1° C. per minute. The resulting siRNA was stored at −20° C. prior to use. The sense oligomer with identity to the endogenous mouse and rat gene encoding cytosolic alanine aminotransferase has the sequence: 5′-rCrArCrUrCrArGrUrCrUrCrUrArArGrG-rGrCrUTT-3′ (SEQ ID 10), which corresponds to positions 928-946 of the cytosolic alanine aminotransferase reading frame in the sense direction. The letter “r” preceding a nucleotide indicates that nucleotide is a ribonucleotide. The sense oligomer with identity to the endogenous mouse and rat gene encoding cytosolic alanine aminotransferase has the sequence: 5′-rArGrCrCrCrUrUrArGrArGrArCrUrGrArGrUrGTT-3′ (SEQ ID 11), which corresponds to positions 928-946 of the cytosolic alanine aminotransferase reading frame in the antisense direction. The letter “r” preceding a nucleotide indicates that nucleotide is a ribonucleotide. The annealed oligomers containing cytosolic alanine aminotransferase coding sequence are referred to as siRNA-ALT

Mice were injected into the tail vein over 7-120 seconds with 40 μg siRNA-ALT diluted in 1-3 ml Ringer's solution (147 mM NaCl, 4 mM KCl, 1.13 mM CaCl₂). Control mice were injected with Ringer's solution without siRNA. Two days after injection, the livers were harvested and homogenized in 0.25 M sucrose. ALT activity was assayed using the Sigma diagnostics INFINITY ALT reagent according to the manufacturers instructions. Total protein was determined using the BioRad Protein Assay. Mice injected with 40 μg siRNA-ALT had an average decrease in ALT specific activity of 32% compared to mice injected with Ringer's solution alone.

### Example 10

Inhibition of expression of virally expressed luciferase in mammalian cells in culture by siRNA. HeLa cells in culture were first infected with adenovirus containing the luciferase gene under control of the phosphoglycerol kinase (PGK) enhancer/promoter (Ad2PGKluciferase). Infection of HeLa cells with Ad2PGKluciferase resulted in expression of luciferase in this cell line. After infection, siRNA targeted to the luciferase coding region or control siRNAs were delivered to the cells and the amount of luciferase activity was determined 24 h later.

HeLa cells were seeded to 50% confluency in Dulbecco's Modified Eagle's Medium (DMEM) supplemented with 10% fetal bovine serum (FBS) in a T25 flask and incubated in a 5% CO₂ humidified incubator at 37° C. 16 h later, cells were washed with PBS, trypsinized, harvested and resuspended in 13 ml DMEM/10% FBS. 500 μl of the cell suspension was distributed to each well in a 24 well plate. After 16 h incubation, the media in each well was replaced with 100 μl DMEM/10% FBS containing 5 μl Ad2PGKluciferase (2.5×10¹⁰ particles/ml stock). After incubation for 2 h, 400 μl DMEM/10% FBS was added to each well followed by the addition of siRNA complexed with TransIT-TKO (Mirus Corporation). For preparation of the siRNA complexes 7.5 μg TransIT-TKO was diluted in 50 μl serum-free Opti-MEM and incubated at room temperature for 5 minutes. siRNA was added in order to give a final concentration of siRNA per well of 0, 1, 10 or 100 nM and incubated for 5 minutes at room temperature. Complexes were then added directly to the wells. SiRNAs targeted to the either luciferase gene, the luciferase⁺ gene, or an unrelated gene product were used (siRNA-Luc, siRNA-Luc⁺, and siRNA-c respectively). Only siRNA-Luc contained sequence identical to Ad2PGKluciferase. All assay points were performed in duplicate wells.

24 hours after delivery of siRNA, cells were lysed and luciferase activity was assayed. Results indicate that luciferase activity was inhibited 35% at 1 nM siRNA-Luc and 53% at 10 nM siRNA-Luc (Table 2). No inhibition was observed using either siRNA-Luc⁺, which contains three base pair mismatches relative to siRNA-luc or siRNA-c. These results demonstrate that siRNA can be used to inhibit expression of a virally encoded gene. In addition, the fact that siRNA-luc⁺ was unable to inhibit luciferase expression demonstrates that siRNA-mediated RNAi exhibits high sequence specificity. This example provides proof-of-principle that siRNA can be used to inhibit the expression of viral gene products in a sequence-specific manner.

TABLE 2
SiRNA-mediated RNA interference of virally encoded
luciferase in HeLa cells.

| | % Luciferase activity | | |
|---|---|---|---|
| [siRNA] | siRNA-Luc | siRNA-Luc⁺ | siRNA-c |
| 0 nM | 100 | NA | NA |
| 1 nM | 65 | 101 | 91 |
| 10 nM | 47 | 117 | 129 |

### Example 11

Delivery of siRNA and morpholino antisense oligonucleotide to mammalian HeLa cells simultaneously. HeLa cells were maintained in Dulbecco's Modified Eagle Medium supplemented with 10% fetal bovine serum. All cultures were maintained in a humidified atmosphere containing 5% CO₂ at 37° C. Approximately 24 hours prior to transfection, cells were plated at an appropriate density in a T75 flask and incubated overnight. At 50% confluency, cells were initially transfected with pGL3 control (firefly luciferase, Promega, Madison Wis.) and pRL-SV40 (sea pansy luciferase, Promega, Madison, Wis.) using TransIT-LT1 transfection reagent according to the manufacturer's recommendations (Mirus Corporation, Madison, Wis.). 15 μg pGL3 control and 50 ng pRL-SV40 were added to 45 μI TransIT-LT1 in 500 μl Opti-MEM (Invitrogen) and incubated 5 min at RT. DNA complexes were then added to cells in the T75 flask and incubated 2 h at 37° C. Cells were washed with PBS, harvested with trypsin/EDTA, suspended in media, plated into a 24-well plate with 250 μl DMEM+10% serum and incubated 2 h at 37° C. After incubation for 2 h, 400 μl DMEM/10% FBS was added to each well followed by the addition of siRNA complexed with TransIT-TKO (Mirus Corporation). For preparation of the siRNA and morpholino-containing complexes, 2 μl TransIT-TKO was diluted in 50 μl serum-free Opti-MEM and incubated at room temperature for 5 minutes. siRNA was added in order to give a final concentration of siRNA per well of 0, 0.1, or 10 nM and morpholino added to give a final concentration of morpholino per well of 0, 10, 100 or 1000 nM and incubated for 5 minutes at room temperature. Complexes were then added directly to the wells. All assay points were performed in duplicate wells.

The pGL3 control plasmid contains the firefly luc+ coding region under transcriptional control of the simian virus 40 enhancer and early promoter region. The pRL-SV40 plasmid contains the coding region for Renilla reniformis, sea pansy, luciferase under transcriptional control of the simian virus 40 enhancer and early promoter region.

Morpholino antisense molecule and siRNAs used in this example were as follows:
Morpholino-Luc (GeneTools Philomath, Oreg.) 5′-TTATGTTTTTGGCGTCTTCCATGGT-3′ (SEQ ID 1; Luc+ −3 to +22 of pGL3 Control Vector), was designed to base pair to the region surrounding the Luc+ start codon in order to inhibit translation of mRNA. Sequence of the start codon in the antisense orientation is underlined.
Standard control morpholino 5′-CCTCTTACCTCAGTTACAATTTATA-3′ (SEQ ID 3), contains no significant sequence identity to Luc+ sequence or other sequences in pGL3 Control Vector
GL3 siRNA-Luc+: SEQ ID 4 and SEQ ID 5.

Single-stranded, gene-specific sense and antisense RNA oligomers with overhanging 3′ deoxynucleotides were prepared and purified by PAGE (Dharmacon, LaFayette, Colo.). The two complementary oligonucleotides, 40 μM each, are annealed in 250 μl 100 nM NaCl 50 mM Tris-HCl, pH 8.0 buffer by heating to 94° C. for 2 minutes, cooling to 90° C. for 1 minute, then cooling to 20° C. at a rate of 1° C. per minute. The resulting siRNA was stored at −20° C. prior to use.

In order to deliver the morpholino to cells in culture using the cationic transfection reagent, TransIT-TKO (Mirus Corporation) the morpholino was first annealed to a DNA oligonucleotide of complementary sequence. The sequence of the DNA strand is as follows: 5′-GCCAAAAACATAAACCATGGAAGACT-3′ (SEQ ID 2). The morpholino and complementary DNA oligonucleotide, 0.5 mM each, are annealed in 5 mM HEPES pH 8.0 buffer by heating to 94° C. for 2 minutes, cooling to 90° C. for 1 minute, then cooling to 20° C. at a rate of 1° C. per minute. The resulting morpholino/DNA complex was stored at −20° C. prior to use.

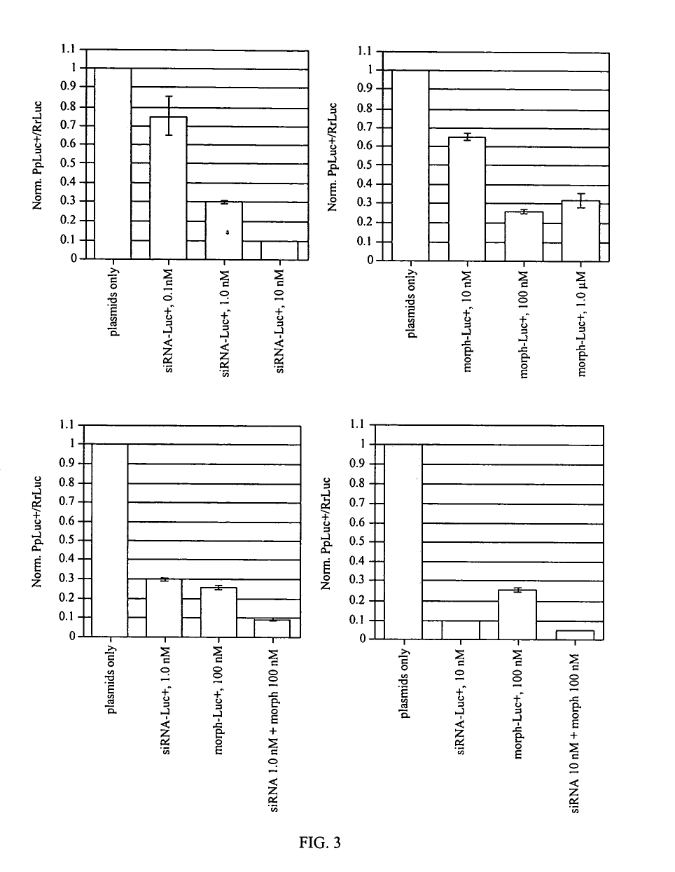

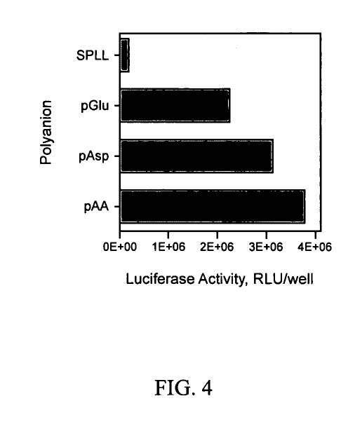

Cells were harvested after 24 h and assayed for luciferase activity using the Promega Dual Luciferase Kit (Promega). A Lumat LB 9507 (EG&G Berthold, Bad-Wildbad, Germany) luminometer was used. The amount of luciferase expression was recorded in relative light units. Numbers were then adjusted for control sea pansy luciferase expression and are expressed as the percentage of firefly luciferase expression in the absence of siRNA (FIG. 3) Numbers are the average for at least two separate wells of cells.

These data demonstrate that when siRNA and morpholino are delivery simultansously, the degree of inhibition is greater than with delivery of either siRNA or morphlino alone.

### Example 12

Inhibition of Luciferase expression by delivery of antisense morpholino and siRNA simultaneously to liver in vivo. Morpholino antisense molecule and siRNAs used in this example were as follows:
DL94 morpholino (GeneTools Philomath, Oreg.), SEQ ID 1 (Luc+ −3 to +22 of pGL3 Control Vector), was designed to base pair to the region surrounding the Luc+ start codon in order to inhibit translation of mRNA. Sequence of the start codon in the antisense orientation is underlined.
Standard control morpholino, SEQ ID 3, contains no significant sequence identity to Luc+sequence or other sequences in pGL3 Control Vector
GL3 siRNA-Luc+: SEQ ID 4 and SEQ ID 5.

DL88:DL88C siRNA (targets EGFP 477-495, nt765-783):

| | |
|---|---|
| 5′-rGrArArCrGrGrCrArUrCrArArGrGrUrGrAr | (SEQ ID 12) |
| ArCdTdT-3′ | |
| 3′-dTdTrCrUrUrGrCrCrCrUrArGrUrUrCrCrAr | (SEQ ID 13) |
| CrUrUrG-5′ | |

Two plasmid DNAs±siRNA and ±antisense morpholino in 1-3 ml Ringer's solution (147 mM NaCl, 4 mM KCl, 1.13 mM CaCl₂) were injected, in 7-120 seconds, into the tail vein of mice. The plasmids were pGL3 control, containing the luc+coding region under transcriptional control of the simian virus 40 enhancer and early promoter region, and pRL-SV40, containing the coding region for the Renilla reniformis luciferase under transcriptional control of the Simian virus 40 enhancer and early promoter region. 2 μg pGL3 control and 0.2 μg pRL-SV40 were injected with or without 5.0 μg siRNA and with or without 50 μg DL94 morpholino. One day after injection, the livers were harvested and homogenized in lysis buffer (0.1% Triton X-100, 0.1M K-phosphate, 1 mM DTT, pH 7.8). Insoluble material were cleared by centrifugation. The homogenate was diluted 10-fold in lysis buffer and 5 μl was assayed for Luc+ and Renilla luciferase activities using the Dual Luciferase Reporter Assay System (Promega Corp.). Ratios of Luc+ to Renilla Luc were normalized to the 0 μg siRNA-Luc+ control.

TABLE 3
Inhibition of luciferase expression from pGL3 control
plasmid in mouse liver after delivery of 50 μg
antisense morpholino, 5 μg siRNA or both.

| Antisense | | percent inhibition of |
|---|---|---|
| morpholino | siRNA | luciferase expression |
| — | — | 0 |
| Standard | DL88:DL88C | 0 |
| DL94 | DL88:DL88C | 85.4 ± 2.7 |
| Standard | GL3 siRNA-Luc+ | 92.0 ± 1.9 |
| DL94 | GL3 siRNA-Luc+ | 98.6 ± 0.5 |

These experiments demonstrate the near complete inhibition of gene expression in vivo when antisense morpholino is delivered together with siRNA. This level if inhibition was greater than that for either morpholino of siRNA individually.

### Example 13

Inhibition of Luciferase expression in lung after in vivo delivery of siRNA using recharged particles. Recharged particles were formed to deliver the reporter genes luciferase+ and Renilla luc as well as siRNA targeted against luciferase+ mRNA or a control siRNA to the lung. In this experiment, particles containing the reporter genes were delivered first, followed by delivery of particles containing the siRNAs. In all cases, particles were prepared with the polycation linear polyethylenimine (IPEI) and the polyanion polyacrylic acid (pAA). For delivery of reporter genes, particles were prepared which contained a mixture of the luc+ and Renilla luc expression plasmids. Normalization of expression of the two luciferase genes corrects for varying plasmid delivery efficiencies between animals. Particles containing a mixture of the expression plasmids containing the luciferase+ gene and the Renilla luciferase gene were injected intravascularly. Particles containing siRNA-Luc+ or a control siRNA were injected intravascularly immediately following injection of the plasmid-containing particles. 24 hours later, the lungs were harvested and the homogenate assayed for both Luc+ and Renilla Luc activity.

Specific experimental details were as follows: plasmid-containing particles were prepared by mixing 45 μg pGL3 control (Luc+) and 5 μg pRL-SV40 (Renilla Luc) with 300 μg IPEI in 10 mM HEPES, pH 7.5/5% glucose. After vortexing for 30 seconds, 50 μg pAA was added and the solution vortexed was for 30 seconds. siRNA-containing particles were prepared similarly, except 25 μg siRNA was used with 200 μg IPEI and 25 μg pAA. Particles containing the plasmid DNAs (total volume 250 μl) were injected into the tail vein of ICR mice. In animals that received siRNA, particles containing siRNA (total volume 100 μl) were injected into the tail vein immediately after injection of the plasmid DNA-containing particles. 1.5 mg pAA in 100 μl was then injected into the tail vein some animal 0.5 h later. 24 h later, animals were sacrificed and the lungs were harvested and homogenized. The homogenate was assayed for Luc+ and Renilla Luc activity using the Dual Luciferase Assay Kit (Promega Corporation).

Results indicate that intravascular injection of particles containing the plasmids pGL3 control and pRL-SV40 results in Luc+ and Renilla Luc expression in lung tissue (Table 2). Injection of particles containing siRNA-Luc+ after injection of the plasmid-containing particles resulted in specific inhibition of Luc+ expression. Renilla Luc expression was not inhibited. Injection of particles containing control siRNA (siRNA-c), targeted against an unrelated gene product did not result in inhibition of either Luc+ or Renilla Luc activity, demonstrating that the effect of siRNA-Luc+ on Luc+ expression is sequence specific and that injection of siRNA particles per se does not generally inhibit delivery or expression of delivered plasmid genes. These results demonstrate that particles formed with lPEI and pAA containing siRNA are able to deliver siRNA to the lung and that the siRNA cargo is biologically active once inside lung cells.

TABLE 5
Delivery of siRNA to the lung using recharged particles
results in inhibition of target gene expression.

| | Relative | | | |
|---|---|---|---|---|
| | light units | | Average Luc+/ | Normalized |
| Particles | Replicate 1 | Replicate 2 | Renilla Luc ratio | Luc+/Renilla Luc |
| plasmids only | | | | |
| Luc+ | 560994 | 680038 | 0.43 +/− 0.05 | 1.00 |
| Renilla Luc | 1406188 | 1452593 | | |
| siRNA-Luc+ | | | | |
| Luc+ | 326697 | 428079 | 0.21 +/− 0.07 | 0.48 +/− 0.16 |
| Renilla Luc | 1283313 | 2683842 | | |
| siRNA-c | | | | |
| Luc+ | 964503 | 1452962 | 0.37 +/− 0.01 | 0.86 +/− 0.03 |
| Renilla Luc | 2527933 | 4005381 | | |

### Example 14

In vivo delivery of siRNA to mouse liver cells using TransIT™ In Vivo. 10 μg pGL3 control and 1 μg pRL-SV40 were complexed with 11 μl TransIT™ In Vivo in 2.5 ml total volume according the manufacturer's recommendation (Mirus Corporation, Madison, Wis.). For siRNA delivery, 10 μg pGL3 control, 1 μg pRL-SV40, and either 5 μg siRNA-Luc+ or 5 μg control siRNA were complexed with 16 μl TransIT™ In vivo in 2.5 ml total volume. Particles were injected over ˜7 s into the tail vein of 25-30 g ICR mice as described in Example 1. One day after injection, the livers were harvested and homogenized as described in Example 1. Luc+ and Renilla Luc activities were assayed using the Dual Luciferase Reporter Assay System (Promega). Ratios of Luc+ to Renilla Luc were normalized to the no siRNA control. siRNA-luc+ specifically inhibited the target Luc+ expression 96% (Table 6).

TABLE 6
Delivery of siRNA to the mouse liver using TransIT ™ In
Vivo results in inhibition of target gene expression.

| | | | | % inhibition of |
|---|---|---|---|---|
| | | expression | relative LUC+ | Luc+ |
| complex | gene | (RLUs) | expression | expression |
| Plasmid alone | Luciferase | 31973057 | 5.1855 | 0.0 |
| | Renilla | 6165839 | | |
| Plasmid + | Luciferase | 853332 | 0.2069 | 96.0 |
| siRNA-Luc+ | Renilla | 4124726 | | |
| Plasmid + | Luciferase | 5152933 | 2.1987 | 57.5 |
| control SiRNA | Renilla | 2343673 | | |

These data show that the TransIT™ In Vivo labile polymer transfection reagent effectively delivers siRNA in vivo.

### Example 15

Inhibition of vaccinia virus in mice. As a model for smallpox infection, the ability to attenuate vaccinia virus infection in mice by siRNA delivery was determined. Groups of 5 mice (C57B1 strain, 4-6 week old) were inoculated by installation of 20 μl of virus in PBS into each nostril with a micropipet, for a total volume of 40 μl containing 10⁴-10⁶ pfu of vaccinia virus (Ankara strain, GenBank accession number U94848), under isoflurane anesthesia. 5 μg E9L DNA polymerase siRNA Sequence 351:

| | |
|---|---|
| 5′-rCrGrGrGrArUrArUrCrUrCrCrArGrArCrGr | (SEQ ID 14) |
| GrAdTdT-3′ | |
| 3′-dTdTrGrCrCrCrUrArUrArGrArGrGrUrCrUr | (SEQ ID 15) |
| GrCrCrU-5′ | |

was delivered at one of several time points relative to viral infection (4 hours before, simultaneous, 4 hours after, 24 hours after, 48 hours after) by injection into tail vein of mice as described above. At 1, 2, 4, and 7 days after infection, mice were sacrificed, tissue sections were collected, and viral load determined in lung, liver, spleen, brain, and bone marrow. Viral pathogenicity was assessed by histology of infected tissues, measurement of viral titers in infected tissues, and mouse survival. Tissue samples embedded in OCT Tissue-Tek were frozen in liquid nitrogen and 10 μm cryosections were fixed in 2% formaldehyde. Following permeabilization with 0.1% Triton X100, sections were blocked and stained with antibodies directed against cell surface markers or viral antigens. Antibodies against CD43 were used to detect infiltrating lymphocytes, as a marker for inflammation and viral pathogenicity. Antibodies directed against vaccinia virus proteins (e.g., A27L) were used to detect sites of viral replication. All antibodies were detected with peroxidase (Vector) or fluorescent (Sigma) secondary reagents. The amount of mRNA of the target gene and control genes were determined using the TaqMan PCR system.

### Example 16

Physiological effects induced by siRNA delivery in vivo—Reduction of serum triglyceride levels using siRNA of HMG CoA reductase in vivo: We have demonstrated a reduction of serum triglyceride levels in mice upon treatment with siRNA directed against HMG CoA reductase. Group A (series2) mice (5 mice) were each injected with 50 μg of an siRNA directed against mouse HMG CoA reductase mRNA. Group B (Series 1) mice (5 mice) were an uninjected control group. Group A and Group B animals were bled 7 days before, 2 days after, 4 days after, and 7 days after the injection. Serum samples were stored at −20° C. until all timepoints had been collected. Each group's serum samples from a given time-point were pooled prior to the triglyceride assays. Triglyceride assays were performed in quintuplicate.

Mice. Experiments were performed in Apoetm1 Unc mice obtained from The Jackson Laboratories (Bar Harbor, Me.). Mice homozygous for the Apoetm1Unc mutation show a marked increase in total plasma cholesterol levels that is unaffected by age or sex. Fatty streaks in the proximal aorta are found at 3 months of age. The lesions increase with age and progress to lesions with less lipid but more elongated cells, typical of a more advanced stage of pre-atherosclerotic lesion. Moderately increased triglyceride levels have been reported in mice with this mutation on a mixed C57BL/6×129 genetic background.

siRNA reagents. Single-stranded, HMG CoA reductase-specific sense and antisense RNA oligomers with overhanging 3′ deoxyribonucleotides were ordered from Dharmacon, Inc. The annealed RNA duplex was resuspended in Buffer A (20 mM KCl, 6 mM HEPES-KOH pH 7.5, 0.2 mM MgCl₂) and stored at −20° C. prior to use. Prior to injection, siRNAs were diluted to the desired concentration (50 μg/2.2 ml) in Ringer's solution.

Oligonucleotide sequences. The sense oligomer with identity to the murine HMG CoA reductase gene has the sequence: 5′-rArCrArUrUrGrUrCrArCrUrGrCrUrArUrCrUrATT-3′ (SEQ ID 25), which corresponds to positions 2324-2344 of the HMG CoA reductase reading frame in the sense direction. The antisense oligomer with identity to the murine HMG CoA reductase gene has the sequence: 5′-rUrArGrArUrArG-rCrArGrUrGrArCrArArUrGrUTT-3′ (SEQ ID 26), which corresponds to positions 2324-2344 of the HMG CoA reductase reading frame in the antisense direction. The letter “r” preceding each nucleotide indicates that nucleotide is a ribonucleotide. The annealed oligomers containing HMG CoA reductase coding sequence are referred to as siRNA-HMGCR.

A total of 50 μg of siRNA-HMGCR was dissolved in 2.2 ml Ringer's solution (147 mM NaCl, 4 mM KCl, 1.13 mM CaCl₂), and injected into the tail vein of ApoE (−/−) mice over 7-12 seconds. Control mice were not injected and are referred to here as naive. Each mouse was bled from the retro-orbital sinus at various times prior to and after injection. Cells and clotting factors were pelleted from the blood to obtain serum. The serum triglyceride levels were then assayed by a enzymatic, colorimetric assay using the Infinity Triglyceride Reagent (Sigma Co.). Results showed that triglyceride levels in siRNA-HMGCR treated mice (Series2) were reduced 62% after two days, 56% after two days, and returned to normal levels after 7 days. No decrease in serum triglyceride levels was observed in uninjected mice (Series1).

Triglyceride assays. Serum samples were diluted 1:100 in the Infinity Triglyceride Reagent (2 μl in 200 μl) in a clear, 96-well plate. Each assay plate was then incubated at 37° C. for five minutes, removed and allowed to cool to room temperature. Absorbance was measured at 520 nm using a SpectraMax Plus plate reader (Molecular Devices, Inc). Background absorbance (no serum added) was subtracted from each reading and the resulted data was plotted versus timepoint.

TABLE 7
Triglyceride levels in animals following delivery of
HMGCR-specific siRNA

Triglyceride levels

| | | siRNA-HMGCR |
|---|---|---|
| day | No Injection Control | Treated Animals |
| −7 | 0.181 ± 0.010 | 0.218 ± 0.008 |
| +2 | 0.194 ± 0.011 | 0.082 ± 0.006 |
| +4 | 0.175 ± 0.012 | 0.095 ± 0.005 |
| +7 | 0.284 ± 0.021 | 0.189 ± 0.012 |

### Example 17

Physiological effects induced by siRNA delivery in vivo—Reduction of PPAR levels using siRNA expression cassettes in vivo: PPARα, peroxisome proliferator-activated receptor α, is a transcription factor and a member of the nuclear hormone receptor superfamily. The gene, found in both mice and humans, plays an important role in the regulation of mammalian metabolism. In particular, PPARα is required for the normal maintenance of metabolic pathways whose misregulation can facilitate the development metabolic disorders such as hyperlipidemia and diabetes. When bound to its ligand, PPARα binds to the retinoid X receptor (RXR) and activates the transcription of genes implicated in maintaining homeostatic levels of serum lipids and glucose. The manipulation of PPARα levels using RNA interference may be a safe and effective way to modulate mammalian metabolism and treat pathogenic hyperlipidemia and diabetes. We used a tail vein injection procedure to delivery plasmid DNA encoding an siRNA expression cassette to modulate endogenous PPARα levels using RNA interference in mice. Our results provide a model for the therapeutic delivery of siRNAs synthesized in vivo from delivered plasmid DNA. This method, or variations thereof, will be generally useful in the modulation of the levels of an endogenous gene using RNA interference.

siRNA hairpin sequences. Initially, we identified a series of plasmid DNA-based siRNA hairpins that exhibited RNAi activity against PPARα in primary cultured hepatocytes. The general hairpin structure consists of a polynucleotide sequence with sense and antisense target sequences flanking a micro-RNA hairpin loop structure. Transcription of the siRNA hairpin constructs was driven by the promoter from the human U6 gene. In addition, the end of the hairpin construct contains five T's to serve as an RNA Polymerase III termination sequence. The siRNA hairpin directed against PPARα had the sequence 5′-GGAGCTTT-GGGAAGAGGAAGGTGTCATCcttcctgtcaGATGGCATCTTCCTCTTCCCGAAGCTCC-TTTTT-3′ (SEQ ID 20). Lower-case letters indicate the sequence of the hairpin loop motif. The entire hairpin construct encoding the PPARα siRNA (consisting of the U6 promoter, the PPARα siRNA hairpin, and the termination sequence) is referred to as pMIR303. The negative control siRNA hairpin directed against GL3 had the sequence 5′-GGATTCCAA-TTCAGCGGGAGCCACCTGATgaagcttgATCGGGTGGCTCTCGCTGAGTTGGAATCC-ATTTTT-3′ (SEQ ID 21). The entire hairpin construct encoding the GL3 siRNA (consisting of the U6 promoter, the GL3 siRNA hairpin, and the termination sequence) is referred to as pMIR277.

Injections of mice. Ten mice in each experimental group were injected three times each with 40 μg/injection of either pMIR277 (GL3 siRNA construct) or pMIR303 (PPARA siRNA construct) using a tail vein injection procedure. Volumes of Ringer's solution (147 mM NaCl, 4 mM KCl, 1.13 mM CaCl₂) corresponding to 10% of each animal's body weight and containing the 40 μg of pMIR277 or pMIR303 were injected into mice over a period of 10 seconds with each injection. For each animal, injection 1 was performed on Day 0, injection 2 was performed on Day 2, and injection 3 was performed on Day 4. Seven days after Injection 3 (Day 11), livers from all mice were harvested and total RNA was isolated using the Tri-Reagent protocol.

Isolation of total RNA and cDNA synthesis. Total mRNA from injected mouse livers was isolated using Tri-Reagent. 500 ng of ethanol precipitated, total RNA suspended in RNase-free water was used to synthesize the first strand cDNA using SuperScript III reverse transcriptase. cDNAs were then diluted 1:50 and analyzed by quantitative, real-time qPCR.

Quantitative, real-time PCR. Bio-Rad's iCycler quantitative qPCR system was used to analyze the amplification of PPARα and GAPDH amplicons in real time. The intercalating agent SYBR Green was used to monitor the levels of the amplicons. Primer sequences used to amplify PPARα sequences were 5′-TCGGGATGTCACACAATGC-3′ (SEQ ID 30) and 5′-AGGCTTCGTGGATTCTCTTG-3′ (SEQ ID 16). Primer sequences used to amplify GAPDH sequences were 5′-CCTCTATATCCGTTTCCAGTC-3′ (SEQ ID 17) and 5′-TTGTCGGTGCAATAGTTCC-3′ (SEQ ID 31). Serial dilutions (1:20, 1:100 and 1:500) of cDNA made from Ringer's control samples were used to create the standard curve from which mRNA levels were determined. PPARα levels were quantitated relative to both GAPDH mRNA and total input RNA.

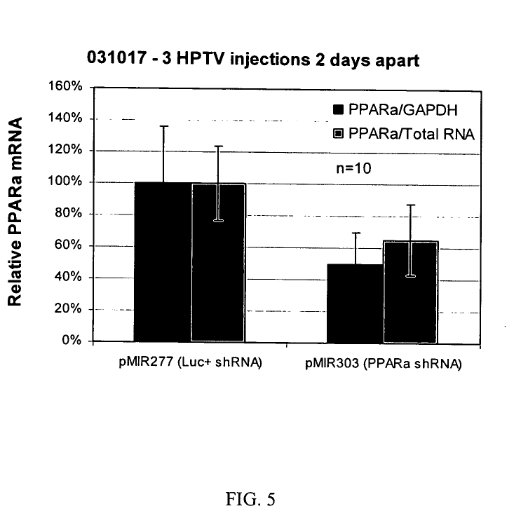

Results: Mouse livers injected with the PPARα hairpin constructs contained 50% or 35% less PPARα mRNA than those injected with GL3 siRNA control hairpins when compared to GAPDH mRNA or total input RNA, respectively. FIG. 5 shows the relative levels of PPARα mRNA as compared to GAPDH mRNA or total input RNA in each 10-mouse group. The experimental error is expressed as the total standard deviation among all samples. That this delivery procedure is able to achieve up to 50% knockdown of an endogenous target transcript demonstrates its general utility for in vivo modulation of gene expression.

### Example 18

Combination therapy using statins and siRNAs for the treatment of hyperlipidemia. Treatment with inhibitors of HMG CoA reductase, commonly known as statins, has been shown to markedly reduce the serum lipid levels of hyperlipidemia patients. Statins inhibit the activity of HMG-CoA reductase. In turn, this inhibition triggers a feedback mechanism through which the cellular levels of HMG-CoA reductase mRNA is markedly upregulated. Here, we present work that demonstrates a significant reduction in the levels of HMGCR mRNA in cells treated with atorvastatin. Addition of bioavailable siRNAs to the treatment regiments of patients on statins will lower the required statin dose, thereby reducing the required dosage of stains and cutting deleterious side effects.

siRNA reagents. Single-stranded, HMG CoA reductase-specific sense and antisense RNA oligomers with overhanging 3′ deoxyribonucleotides were ordered from Dharmacon, Inc. The annealed RNA duplex was resuspended in Buffer A (20 mM KCl, 6 mM HEPES-KOH pH 7.5, 0.2 mM MgCl₂) and stored at −20° C. prior to use. Prior to injection or transfection, siRNAs were diluted to the desired concentration (50 μg/2.2 ml) in Ringer's solution or (25 nM) in OPTI-MEM/Transit-TKO, respectively.

Oligonucleotide sequences. The sense oligomer with identity to the murine HMG CoA reductase gene has the sequence: SEQ ID 25, which corresponds to positions 2324-2344 of the HMG CoA reductase reading frame in the sense direction. The antisense oligomer with identity to the murine HMG CoA reductase gene has the sequence: SEQ ID 26, which corresponds to positions 2324-2344 of the HMG CoA reductase reading frame in the antisense direction. The letter “r” preceding each nucleotide indicates that nucleotide is a ribonucleotide. The annealed oligomers containing HMG CoA reductase coding sequence are referred to as siRNA-HMGCR.

A total of 50 μg of siRNA-HMGCR was dissolved in 2.2 ml Ringer's solution (147 mM NaCl, 4 mM KCl, 1.13 mM CaCl₂), and injected into the tail vein of mice over 7-120 seconds. Control mice were not injected and are referred to here as naive.

qPCR assays. Quantitative, real-time PCR was performed using the Bio-Rad icycler system and iCycler reagents as recommended by the manufacturer. The primers used to amplify HMGCR sequences were SEQ ID 17 and SEQ ID 31.

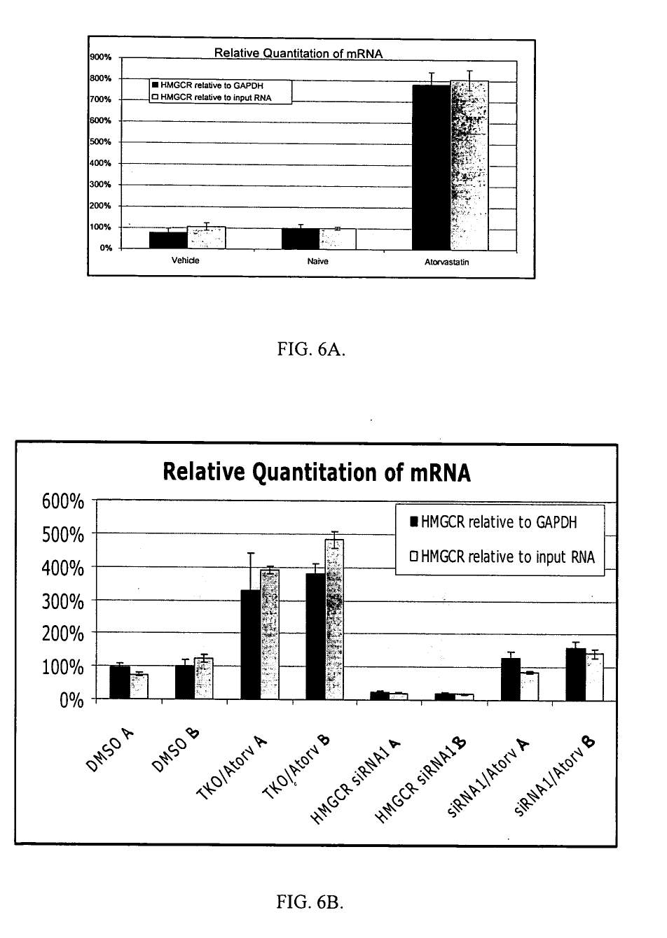

RESULTS Induction of HMG-CoA reductase in vivo. C57B6 mice treated for 48 hours with a 50 mg/kg dose of atorvastatin showed an expected and marked increase in HMG-CoA reductase mRNA levels as measured by quantitative, real-time PCR (FIG. 6A). Livers from groups of 10 mice were harvested 48 hours after treatment was commenced and pooled mRNA populations (10 mice/pool) were assayed for HMGCR levels. Mice treated with atorvastatin had, on average, an 800% increase in HMGCR mRNA.

Prevention of atorvastatin-induced upregulation of HMG-CoA reductase mRNA. As shown above, atorvastatin treatment results in a marked increase in the amount of HMGCR mRNA present in the livers of mice. Primary hepatocytes were isolated from C57B6 mice and cultured for 24 hours in the presence or absence of anti-HGMCR siRNAs and 10 μm atorvastatin. Total RNA from these cells was isolated and transcribed into cDNA using an oligo-dT primer and reverse transcriptase. Subsequently, HMGCR levels were assayed using quantitative, real-time PCR. HMGCR mRNA levels were induced 400% relative to vehicle-treated cells after 24 hours of exposure to atorvastatin (FIG. 6B). Simultaneous administration of the anti-HMGCR siRNA along with the statin held HMGCR levels to those seen in vehicle-treated controls. In addition, treatment of hepatocytes with the HMGCR-directed siRNA alone resulted in the knockdown of HMGCR mRNA to approximately 20% of that seen in control cells. These results show that the simultaneous delivery of an siRNA against HMGCR to cells treated with an HMGCR inhibitor can reduce the relative level of HMGCR mRNA to wild type levels seen in control cells. This strategy should reduce the amount of drug needed to inhibit cellular HMGCR and potentially lower the dose of drug needed in target validation or therapeutic applications in this and other protein families.

### Example 19

Combination therapy using statins and siRNAs for the treatment of hyperlipidemia in vivo. Initially, we identified a series of siRNAs that exhibited RNAi activity against PPARα in primary cultured hepatocytes. Having identified several highly active siRNAs, we selected one to use in our in vivo demonstration of siRNA delivery. siRNA sequences. All RNA sequences were ordered from Dharmacon, Inc. The siRNA duplex directed against PPARα contained the target sequence 5′-rGrArTrCrGrGrArGrCrT-rGrCrArArGrArTrTrC-3′ (SEQ ID 28). A control GL3 siRNA duplex contained the target sequence 5′-rArArCrUrUrArCrGrCrUrGrArGrUrArCrUrUrCrGrA-3′ (SEQ ID 24). The “r” between each indicated base is used to indicate that the oligonucleotides are oligoribo-nucleotides. All siRNAs contained dTdT overhangs.

Injections of mice. Four mice in each experimental group were injected with 50 μg of siRNA using the high-pressure tail vein procedure. A volume of Ringer's solution (147 mM NaCl, 4 mM KCl, 1.13 mM CaCl₂) corresponding to 10% of each animal's body weight and containing 50 μg of PPARα siRNA sequences (or controls) were injected into mice over a period of 10 seconds. After 48 hours, livers from injected mice were harvested and total RNA was isolated.

Isolation of total RNA and cDNA synthesis. Total mRNA from injected mouse livers was isolated using Tri-Reagent. 500 ng of ethanol precipitated, total RNA suspended in RNase-free water was used to synthesize the first strand cDNA using SuperScript III reverse transcriptase. cDNAs were then diluted 1:50 and analyzed by quantitative, real-time qPCR.

Quantitative, real-time PCR. Bio-Rad's iCycler quantitative qPCR system was used to analyze the amplification of PPARα and GAPDH amplicons in real time. The intercalating agent SYBR Green was used to monitor the levels of the amplicons. Primer sequences used to amplify PPARα sequences were SEQ ID 30 and SEQ ID 16. Primer sequences used to amplify GAPDH sequences were SEQ ID 17 and SEQ ID 31. Primers used to amplify PTEN sequences were 5′-GGGAAGTAAGGACCAGAGAC-3′ (SEQ ID 23) and 5′-ATCATCTTGTGAAACAGCAGTG-3′ (SEQ ID 18). Serial dilutions (1:20, 1:100 and 1:500) of cDNA made from Ringer's control samples were used to create the standard curve from which mRNA levels were determined.

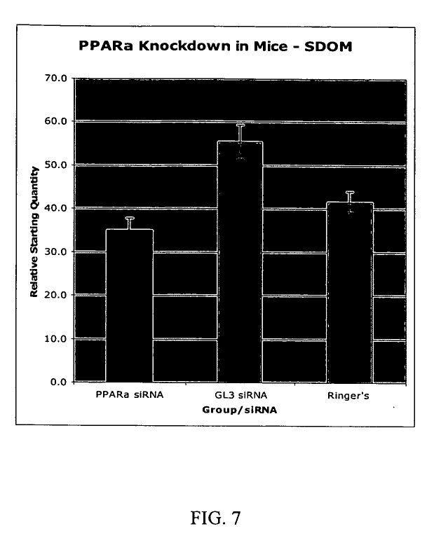

RESULTS: Mouse livers injected with siRNAs directed against PPARa contained 17% or 37% less PPARa mRNA than Ringer's control or GL3 siRNA control animals, respectively. FIG. 7 shows the relative levels of PPARa mRNA as compared to total input RNA in each four-mouse group. The experimental error is expressed as the standard deviation of the mean.

### Example 20

Combination treatment to reduce LDL-cholesterol levels in liver cells. Treatment with inhibitors of HMG-CoA Reductase, commonly known as statins, has been shown to markedly reduce serum LDL-cholesterol levels in hyperlipidemia patients. Statins inhibit the enzymatic activity of HMG-CoA Reductase. Inhibition of HMG-CoA Reductase causes decreased levels of cholesterol biosynthesis. To compensate for the reduced levels of cholesterol synthesis occurring in cells treated with statins, the low density lipoprotein receptor (LDLR) is upregulated through a specific, SREBP-dependent mechanism that senses the effective levels of cholesterol in cellular membranes. This upregulation of the LDLR results in increased cellular uptake of LDL-cholesterol and is one mechanism through which statins may exert their lipid-lowering effects. However, inhibition of cholesterol biosynthesis also triggers a feedback mechanism through which the cellular levels of HMG-CoA Reductase mRNA is markedly upregulated.

When HMG-CoA reductase activity drops below a certain threshold, the cell compensates by upregulating the LDL receptor, bringing cholesterol into the cell to replace the depleted endogenous stores. LDL receptor upregulation can be used as an indicator that HMG-CoA reductase activity had dropped below this threshold. We demonstrate that the levels of HMG-CoA reductase activity can be reduced by cotreatment with both statins and siRNA.

siRNA reagents. Single-stranded, HMG CoA reductase-specific sense and antisense RNA oligomers with overhanging 3′ deoxyribonucleotides were synthesized (Dharmacon, Inc). These single-stranded oligomers were annealed by stepwise cooling of a solution of the oligos from 96° C. to 15° C. The annealed RNA duplex was resuspended in Buffer A (20 mM KCl, 6 mM HEPES-KOH pH 7.5, 0.2 mM MgCl₂) and stored at −20° C. prior to use. Prior to transfection, siRNAs were diluted to the desired concentration (25 nM) in OPTI-MEM/TransIT-TKO (Mirus, Inc).

Oligonucleotide sequences. The antisense oligomer with identity to the murine HMG CoA reductase gene has the sequence: 5′-rCrCrArCrArArArUrGrArArGrArCrUrUrArUrATT-3′ (SEQ ID 27), which corresponds to positions 2793-2812 of the HMG CoA reductase reading frame in the sense direction. The antisense oligomer with identity to the murine HMG CoA reductase gene has the sequence: 5′-rUrArUrArArGrUrCrUrUrCrArUrUrUrGrUrGrGTT-3′ (SEQ ID 29), which corresponds to positions 2793-2812 of the HMG CoA reductase reading frame in the sense direction. The letter “r” preceding a nucleotide indicates that the nucleotide is a ribonucleotide. The annealed oligomers containing HMG CoA reductase coding sequence are referred to as siRNA-HMGCR.

Transfection and atorvastatin treatment of hepatocytes. Just prior to the addition of siRNA transfection cocktails (see below), fresh hepatocyte maintenance media supplemented with various concentrations of atorvastatin was added to each well in a 12-well, collagen coated plate that had been seeded with primary hepatocytes 24 hours previously. Then 100 μl of the siRNA transfection cocktail was added to each well. Hepatocyte maintenance media was a 1:1 mixture of DMEM-F12/0.1% BSA/0.1% galactose.

siRNA transfection cocktail. Each 100 μl aliquot of siRNA transfection cocktail contained 3.8 μl TransIT-TKO, 275 nM siRNA, and the remaining volume of OPTI-MEM transfection media. The 100 μl aliquots were added to cells in 1 ml of media such that the final siRNA concentration was 25 nM.

RNA isolation. After 24 hours of siRNA transfection and atorvastatin treatment, cells were harvested in Tri-Reagent. RNA was isolated, quantitated, and corresponding cDNAs from an oligo-dT primer were synthesized with reverse transcriptase.

qPCR assays. Quantitative, real-time PCR was performed using the Bio-Rad iCycler system and iCycler reagents as recommended by the manufacturer. The primers used to amplify LDLR sequences were 5′-GCATCAGCTTGGACAAGGTGT-3′ (SEQ ID 19) and 5′-GGGAACAGCCACCATTGTTG-3′ (SEQ ID 22).

Primary hepatocytes were isolated from C57BL6 mice and plated on collagen-coated 12-well plates. After allowing them to adhere to the plates for 24 hours, one of two different procedures was followed. In the first, cells were treated with 200 nM atorvastatin in DMSO or DMSO alone for 24 hours. In the second, cells were covered with 1 ml of hepatocyte maintenance media. Next, 100 μl of an siRNA (HMGCR or GL3 control) cocktail (see above) was added to each well such that the final concentration of atorvastatin was 200 nM, 100 nM, 50 nM, 25 nM, or 0 nM and the final concentration of siRNA was 25 nM. Cells were incubated in the atorvastatin/siRNA mixture for 24 hours. Following all 24-hour incubations, cells were harvested in Tri-Reagent and processed for qPCR as described above.

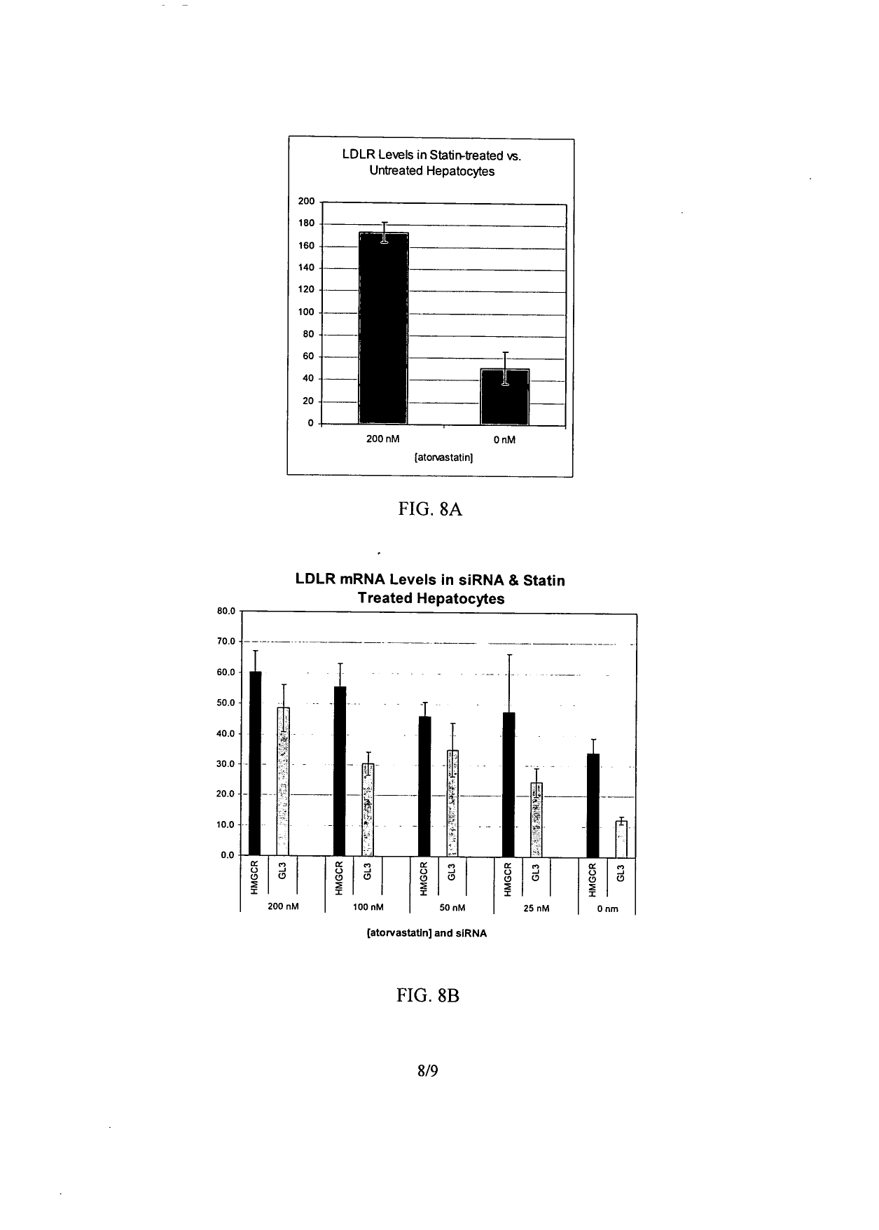

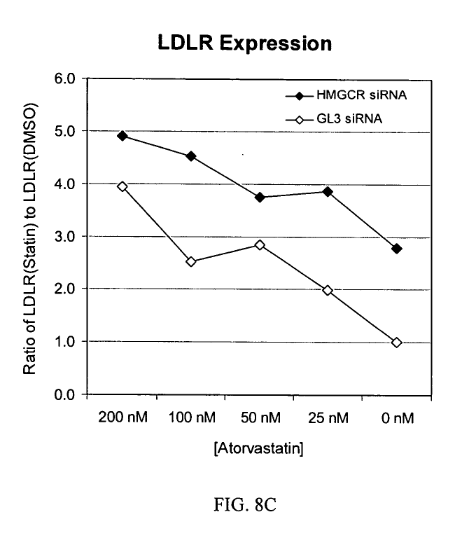

Induction of the LDL receptor in primary murine hepatocytes. Primary hepatocytes isolated by perfusion of C57BL6 mice and treated with 200 nM atorvastatin for 24 hours showed a marked increase in LDL receptor mRNA levels as measured by quantitative, real-time PCR (FIG. 8A).

This result shows the expected upregulation of LDLR mRNA upon treatment with atorvastatin. Next, we treated isolated hepatocytes with a range of atorvastatin concentrations and measured the amount of LDLR mRNA in each sample. In addition, cells were transfected with siRNAs against HMG-CoA reductase or control siRNA against luciferase (GL3). In each case, atorvastatin triggered a dose-dependent increase in LDLR mRNA levels (FIG. 8B). Furthermore, addition of siRNA to the cells further increased LDLR levels.

The data in FIG. 8B indicate that cells treated with HMGCR siRNAs required lower doses of atorvastatin to achieve a corresponding level of LDLR upregulation. For example, one can compare HMGCR siRNA-treated cells exposed to 25 nm or 50 nM statin with GL3 siRNA-treated cells exposed to 200 mM statin and see a similar level of LDLR mRNA was present in those cells. In addition, FIG. 8B indicates that simply reducing the amount of HMGCR in the cell results in an approximately 3-fold upregulation of LDLR mRNA (0 nM atorvastatin lanes). This shows that the HMGCR siRNA alone is effective in reducing cellular HMG-CoA reductase activity and thus increasing LDLR levels.

We used cells treated with GL3 siRNA and 0 nM atorvastatin as a baseline to compare the upregulation of LDLR mRNA in the other samples. The relative starting quantity of LDLR mRNA in each sample was plotted relative to the “baseline” LDLR mRNA level seen in GL3/no statin cells (FIG. 8C). The plot in FIG. 8C clearly shows that lower doses of atorvastatin were necessary to get comparable statin/no statin ratios in cells treated with HMGCR siRNAs.

In summary, we have demonstrated that siRNAs can be used to lower the effective dose of a small molecule inhibitor directed against the product of a gene targeted by the siRNA. This technology has applications in small molecule combination therapies as well as in drug discovery and research applications. For example, using siRNAs to decrease the gene dosage in cells being screened with small molecule libraries can sensitize cell-based assays and make otherwise difficult to detect cellular phenotypes apparent.

The principle demonstrated here can be applied to situations in which the target of the small molecule and the siRNA are not the same. For example, a small molecule inhibitor of a protein required for the efflux of cellular cholesterol (e.g., ABCA1), coupled with an siRNA against HMGCR mRNA, could work together to lower the levels of total serum cholesterol. This would be expected to result in the upregulation of the LDL receptor and a corresponding increase in LDL-C uptake. In addition, G-protein coupled receptor (GPCR) mediated signaling pathways could be modulated by simultaneously treating cells with GPCR antagonists and siRNAs targeting the second messenger pathways within cells.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

1. A process for therapeutic treatment of a disease in a mammal comprising:
a) making a polynucleotide-based gene expression inhibitor containing a sequence that is substantially complementary to a nucleic acid sequence in a gene in said mammal;
b) inserting said polynucleotide-based gene expression inhibitor into a vessel in said mammal;
c) increasing the permeability of said vessel; and,
d) delivering said polynucleotide-based gene expression inhibitor to parenchymal cells in said mammal wherein said polynucleotide-based gene expression inhibitor is available to inhibit expression of said gene.

2. The process of claim 1 wherein said polynucleotide-based gene expression inhibitor consists of siRNA.

3. The process of claim 1 wherein said polynucleotide-based gene expression inhibitor consists of an siRNA expression vector.

4. The process of claim 1 wherein said disease consists of a metabolic disorder.

5. The process of claim 1 wherein said metabolic disorder consists of hyperlipidemia.

6. The process of claim 1 wherein said metabolic disorder consists of diabetes.

7. The process of claim 1 wherein said gene consists of peroxisome proliferator-activated receptor α.

8. A process for altering the endogenous properties of a cell comprising: delivering to said cell a small molecule drug and an polynucleotide-based gene expression inhibitor.

9. The process of claim 8 wherein said polynucleotide-based gene expression inhibitor is selected from the group consisting of: siRNA and siRNA expression vector.

10. The process of claim 9 wherein said small molecule drug and said polynucleotide-based gene expression inhibitor affect the activity of a single gene

11. The process of claim 10 wherein said small molecule drug and said polynucleotide-based gene expression inhibitor affect the activity of different genes.

12. The process of claim 9 wherein delivery of said polynucleotide-based gene expression inhibitor enhances effectiveness of said small molecule drug.

13. The process of claim 12 wherein delivery of said polynucleotide-based gene expression inhibitor reduces a dosage of said small molecule drug required to achieve a therapeutic effect.

14. The process of claim 8 wherein said small molecule drug consists of a statin.

15. The process of claim 14 wherein said polynucleotide-based gene expression inhibitor consists of a HMG CoA reductase-specific siRNA.

16. The process of claim 14 wherein said polynucleotide-based gene expression inhibitor consists of a PPARα-specific siRNA.

17. A process for enhancing effectiveness of a small molecule drug in a mammal comprising: delivering to said mammal a polynucleotide-based gene expression inhibitor.

18. The process of claim 17 wherein said polynucleotide-based gene expression inhibitor is selected from the group consisting of: siRNA and siRNA expression vector.

19. The process of claim 18 wherein said small molecule drug and said polynucleotide-based gene expression inhibitor affect the activity of a single gene

20. The process of claim 18 wherein said small molecule drug and said polynucleotide-based gene expression inhibitor affect the activity of different genes.
